(19) United States
(12) Patent Application Publication
(10) Pub. No.: US 2023/0405200 A1
(43) Pub. Date: Dec. 21, 2023

(54) Radial Access Systems and Methods for Delivery of Gas-Enrichment Therapy

(71) Applicant: Zoll Circulation, Inc., San Jose, CA (US)

(72) Inventors: Ramu Perumal, Gibsonia, PA (US); Jeffrey Lance Creech, Los Angeles, CA (US); Stephen Elwood Myrick, Tustin, CA (US); Gary A. Freeman, Waltham, MA (US); Richard A. Helkowski, Redwood City, CA (US); Fred Shen, San Carlos, CA (US)

(21) Appl. No.: 18/445,076

(22) Filed: Mar. 29, 2023

Related U.S. Application Data
(60) Provisional application No. 63/324,726, filed on Mar. 29, 2022.

(51) Int. Cl. A61M 1/32; A61M 1/36; A61M 25/00; A61M 25/01

(52) U.S. Cl. CPC A61M 1/32; A61M 1/3621; A61M 25/0021; A61M 25/01; A61M 2005/006

(57) ABSTRACT

Methods and systems for delivering gas-enriched blood within a vasculature of a patient may include providing a gas-enrichment system, the gas-enrichment system comprising a mixing chamber and a blood pump. The process may include inserting a catheter for drawing blood from the patient into a radial artery of the patient. The process may include drawing blood from the radial artery or from a vessel upstream of the radial artery at a blood flow rate without collapsing the artery or vessel to a degree that would substantially impede drawing blood. The process may include generating a gas-enriched blood by mixing the withdrawn blood with a gas-enriched liquid in a mixing chamber. The process may include delivering the gas-enriched blood to the vasculature of the patient.

### CLAIM OF PRIORITY

This application claims priority under 35 U.S.C. § 119(e) to U.S. patent application Ser. No. 63/324,726, filed on Mar. 29, 2022, the entire contents of which are hereby incorporated by reference.

### TECHNICAL FIELD

The disclosure relates to systems and methods for the delivery of gas-enriched blood into a patient.

### BACKGROUND

Gas-enriched liquids are desirable in a wide variety of applications. However, at ambient pressure, the relatively low solubility of many gases, such as oxygen or nitrogen, within a liquid, such as water, produces a relatively low concentration of the dissolved gas in the liquid. One method of obtaining an increase in the gas concentration level without significant increase in liquid volume involves an injection and mixing of a gas-enriched liquid into a liquid of interest. A liquid can be gas-enriched at high pressure.

Conventional methods for the delivery of oxygenated blood or oxygen-enriched liquids to tissues and bodily liquids involve the use of extracorporeal circuits for blood oxygenation. Extracorporeal circuits require withdrawing blood from a patient, circulating the blood through an oxygenator to increase blood oxygen concentration, and then delivering the blood back to the patient.

### SUMMARY

This document describes a gas-enrichment system configured to deliver gas-enriched blood intravenously to a patient. The system for delivering gas-enriched blood within the vasculature of a patient (hereinafter the delivery system) is configured to connect to a catheter device to deliver the gas-enriched blood to the patient. The delivery system includes a blood circuit having a draw line and a return line. The draw line and return line are configured to connect to the catheter. Blood is withdrawn from the patient via the draw line. The blood is mixed with a gas-enriched liquid, or oxygen-enriched liquid such as a supersaturated oxygen (SSO₂) enriched liquid, to create gas-enriched blood or supersaturated oxygen (SSO2) enriched blood. The gas-enriched blood is delivered back to the patient through the catheter via the return line, e.g., to provide localized delivery of gas enriched blood to ischemic tissue in the patient. For example, SSO2 therapy may deliver gas-enriched arterial blood directly to at-risk or ischemic myocardial tissue, increasing oxygen diffusion to the ischemic zone, thereby reducing endothelial swelling in the microvasculature and restoring microvascular flow

The delivery systems described herein are configured to deliver gas-enriched liquid (e.g., gas-enriched blood) to the vasculature of the patient. The blood circuit may include a blood circuit in which a catheter connected to a blood draw line is inserted into a radial artery, providing radial access to the vasculature, such that blood is drawn from the radial vasculature (e.g., radial arteries) of the patient or other vessels via the radial vasculature. The delivery system is configured to allow blood draw from the radial artery and/or from a vessel upstream of the radial artery in a precise and controlled manner to avoid collapse of the radial artery or vessel upstream of the radial artery. The radial artery includes a blood vessel that supplies blood to the forearm (lower part of the arm) and hand. The radial artery generally runs along a radial aspect of an anterior compartment of the forearm under the brachioradialis, lateral to the flexor carpi radialis tendon. For the distal section of its course, the radial artery lies on the surface of the radius. In certain implementations, the blood circuit may include a bi-radial blood circuit in which a catheter connected to a blood draw line and a catheter connected to a blood return line are both inserted into respective radial arteries of the patient, providing bi-radial access to the vasculature, such that blood is drawn from the radial artery or from another vessels upstream of the radial artery, e.g., brachial artery, axillary artery, or subclavian artery, via the radial vasculature and returned to the radial vasculature (e.g., radial arteries) or other vessels upstream of the radial artery, e.g., brachial, axillary, or subclavian artery, of the patient.

The systems and methods described herein provide one or more advantages. The delivery system is configured to allow blood draw from the radial artery or other vessel upstream of the radial artery, e.g., brachial, axillary, or subclavian artery, in a precise and controlled manner to avoid collapse of the radial artery and/or other vessel upstream of the radial artery, e.g., brachial, axillary, or subclavian artery. Collapse may refer to a crumpling or sagging of artery walls of the artery causing blood flow blockage in the artery. In certain examples, collapse of an artery may refer to excessive flow causing high shear stress, which in turn causes vasospasm and abrupt closure (spasm) of the vessel. Though artery collapse can occur due to stenosis or other causes, in the context of SSO₂ therapy, collapse of the artery can occur due to low internal pressure in the artery responsive to a blood draw rate exceeding a threshold rate. The threshold flow rate is relatively small in the radial artery relative to the aortal artery or subclavian artery, and therefore a maximum blood draw rate from the radial artery may be lower than the aortal artery or subclavian artery.

The delivery systems described herein enable blood draw and return through access points in the radial arteries. This allows localized access to the vasculature of the patient (e.g., at the radial artery) without requiring insertion of catheters in other arteries or accessing other arteries of the patient (e.g., femoral artery, aorta, etc.). The localized access to the vasculature simplifies the blood circuit. For example, using the radial artery to access the patient's vasculature can reduce bleeding compared to accessing the femoral artery. The simplified process can reduce a time to discharge of the patient compared to a system that accesses the vasculature via the femoral artery or other arteries away from the radial artery. The delivery system allows gas enrichment therapy such as SSO2 therapy to be performed without reducing a mobility of the patient because the femoral artery can be left untouched. The radial access to the vasculature of the patient reduces infection rates and bleeding and is easier to access and easier to close when gas enrichment therapy such as SSO2 therapy is completed. For example, it's standard practice to provide access to the vasculature via the radial artery for other procedures, such as angioplasty and percutaneous Intervention (PCI), and such access can be performed by various caregivers, such as doctors and nurses, allowing of ease and convenience of setup of the blood circuit for performing gas-enrichment therapy, such as SSO2 therapy.

In some implementations, the delivery system enables the draw line to be inserted into a radial vasculature of the patient and advanced to a vessel upstream of the radial artery, e.g., the brachial, axillary, or subclavian artery. The subclavian arteries includes a pair of large arteries in the thorax that supply blood to the thorax itself and the head, neck, shoulder, and arms of the patient. Depending on the side of the body, the subclavian artery can have two origins: the aortic arch on the left and the brachiocephalic trunk on the right. The draw line catheter can be advanced into the subclavian artery to allow for a larger quantity of blood to be drawn from the patient at a faster draw flow rate (e.g., without collapsing the artery) compared to the radial artery. Advancing the draw line to the subclavian artery allows for draw line access through the radial artery and thus localized access to the vasculature of the patient without requiring access to the femoral artery, aorta, or other portion of the vasculature of the patient.

One or more of the advantages are enabled by one or more of the following embodiments.

In a general aspect, a method for delivering gas-enriched blood within a vasculature of a patient includes providing a gas-enrichment system, the gas-enrichment system comprising a mixing chamber and a blood pump. The method includes inserting a catheter for drawing blood from the patient into a radial artery of the patient. The method includes drawing blood from the radial artery or from a vessel upstream of the radial artery at a blood flow rate without collapsing the artery or vessel to a degree that would substantially impede drawing blood. The method includes generating a gas-enriched blood by mixing the withdrawn blood with a gas-enriched liquid in the mixing chamber. The method includes delivering the gas-enriched blood to the vasculature of the patient.

In some implementations, the catheter is advanced to the vessel upstream of the radial artery and the blood is drawn from the vessel upstream of the radial artery.

In some implementations, the blood flow rate is a predetermined blood flow rate of 10-500 mL/min, 30-300 mL/min, or 50 -150 mL/min.

In some implementations, an inner diameter and length of the catheter are sufficient to support a predetermined blood flow rate of 50-150 ml/min while avoiding a pressure drop that would cause pump cavitation. In some implementations, the inner diameter is 6-7 French, the length is 10 to 100 cm and the pressure drop is from 0 mmHG to −100 mmHG.

In some implementations, inserting the catheter into a radial artery of the patient includes accessing a subclavian artery of the patient through the radial artery, and advancing the catheter into the subclavian artery for drawing the blood from the subclavian artery.

In some implementations, inserting the catheter into the vasculature of the patient includes inserting a sheath into the vasculature of the patient, the sheath configured to support the catheter in the vasculature of the patient, and inserting the catheter into the sheath.

In some implementations, the sheath comprises a braided wire and a plastic liner over the braided wire.

In some implementations, the sheath is between 50-100 centimeters in length.

In some implementations, inserting the catheter into a radial artery of the patient includes advancing the catheter into the radial artery of the patient until distal band on the catheter aligns with a predetermined location in the vasculature of the patient.

In some implementations, the process includes controlling a draw rate of the catheter. The controlling includes determining a maximum draw rate based on a size of the radial artery; determining a minimum draw rate and a draw pressure based on a pump flow requirement of a pump configured to draw the blood from the radial artery; and controlling the draw rate to be between the maximum draw rate and the minimum draw rate.

In some implementations, the maximum draw rate prevents controlling a given draw rate causing a collapse of the radial artery, and wherein the minimum draw rate prevents controlling a given draw rate causing cavitation of the pump.

In some implementations, the draw rate is a function of a length of the catheter.

In some implementations, the minimum draw rate is 100 milliliters (ml) per minute and wherein the draw pressure is at least 50 millimeters per Mercury (mmHg).

In some implementations, the process includes measuring the draw rate using a flow sensor; and generating an alert in response to measuring, by the flow sensor, that the draw rate is greater than the maximum draw rate or is less than the minimum draw rate.

In some implementations, the process includes measuring the draw pressure using a pressure sensor; and generating an alert in response to measuring, by the pressure sensor, that the draw pressure is greater than a maximum draw pressure.

In some implementations, one or more lumens of the catheter comprise a braided pattern.

In some implementations, the braided pattern comprises a rectangular cross section.

In some implementations, one or more lumens of the catheter each comprise a wall thickness between 0.005 inches to 0.015 inches, the wall thickness preventing kinking of the one or more lumens of the catheter.

In some implementations, one or more lumens of the catheter comprise an atraumatic tip.

In some implementations, the gas-enriched blood is formed in the mixing chamber by mixing the blood withdrawn from the patient with the gas-enriched liquid generated by a gas enrichment chamber.

In some implementations, the gas-enriched liquid comprises a supersaturated oxygen liquid. In some implementations, the supersaturated oxygen liquid has an O₂ concentration of 0.1-6 ml O₂/ml liquid (STP).

In some implementations, the gas-enriched blood comprises a supersaturated oxygen enriched blood. In some implementations, the supersaturated oxygen enriched blood comprises a supersaturated oxygen enriched blood having a pO2 of 600-1500 mmHg.

In some implementations, the process includes inserting a second catheter into a second radial artery of the patient for delivering the gas-enriched blood to the vasculature of the patient.

In some implementations, the process includes measuring a blood pressure in the radial artery or a vessel upstream of the radial artery using one or more pressure sensors, wherein a controller of the gas-enrichment system receives a signal from the one or more pressure sensors.

In some implementations, the controller generates an alert in response to receiving a signal from the pressure sensor indicating a blood pressure or change in blood pressure that exceeds a threshold or is below a threshold.

In some implementations, the controller controls a pump to adjust the blood draw flow rate in response to receiving a signal from the pressure sensor indicating a blood pressure or change in blood pressure that exceeds a threshold or is below a threshold.

In some implementations, the vessel upstream of the radial artery is the brachial, axillary, or subclavian artery.

In some implementations, drawing blood without collapsing the artery or vessel to a degree that would substantially impede drawing blood comprises preventing a collapse that would result in more than 5-10% reduction in cross-sectional area of an artery or vessel. In some implementations, the catheter comprises a coiled lumen configured to prop open a vessel of the patient. In some implementations, the catheter comprises a straight lumen. In some implementations, the catheter comprises a coiled lumen configured to prop open a vessel of the patient. In some implementations, the catheter comprises a straight configuration upon insertion and assumes a coiled configuration inside the artery or vessel.

In a general aspect, a system for delivering gas-enriched blood within a vasculature of a patient includes a blood circuit. The blood circuit includes a pump configured to circulate blood in the blood circuit; a mixing chamber configured to mix blood of the patient with a gas-enriched liquid to form a gas-enriched blood; a catheter; and a draw line coupled to the mixing chamber and configured to connect the catheter to the mixing chamber. The catheter is configured to be inserted into a radial artery of the patient, the catheter comprising one or more lumens configured to draw the blood from the radial artery or from a vessel upstream of the radial artery at a blood flow rate without collapsing the artery or vessel to a degree that would substantially impede drawing blood and send the blood to the mixing chamber.

In some implementations, the catheter is advanced to the vessel upstream of the radial artery and blood is drawn from the vessel upstream of the radial artery.

In some implementations, the blood flow rate is a predetermined blood flow rate of 10-500 mL/min, 30-300 mL/min, or 50 -150 mL/min.

In some implementations, an inner diameter and length of the catheter are sufficient to support a predetermined blood flow rate of 50-150 ml/min while avoiding a pressure drop that would cause pump cavitation.

In some implementations, the inner diameter is 6-7 French, the length is 10 to 100 cm and the pressure drop is from 0 mmHG to negative 100 mmHG.

In some implementations, the catheter is configured for insertion into a subclavian artery of the patient through the second radial artery.

In some implementations, the one or more lumens of the catheter comprise a braided pattern.

In some implementations, the braided pattern comprises a rectangular cross section.

In some implementations, the one or more lumens of the catheter each comprise a wall thickness between 0.005 inches to 0.015 inches, the wall thickness preventing kinking of the one or more lumens of the catheter.

In some implementations, the one or more lumens of the catheter comprise an atraumatic tip.

In some implementations, the system includes a sheath configured for inserting into the radial artery of the patient, the sheath configured to support the catheter in radial artery. In some implementations, the sheath comprises a braided wire and a plastic liner over the braided wire. In some implementations, the sheath is between 50-100 centimeters in length.

In some implementations, the catheter comprises a distal band on a distal portion of the catheter, the distal band configured to align with a predetermined location in the radial artery of the patient.

In some implementations, the system includes one or more flow sensors for measuring the draw rate; and a controller configured to receive a signal from the one or more flow sensors, wherein the controller is configured to generate an alert in response to receiving a signal from the flow sensor indicating that the draw rate is greater than a maximum draw rate or is less than a minimum draw rate.

In some implementations, the system includes one or more pressure sensors for measuring the blood pressure in the radial artery; and a controller configured to receive a signal from the one or more pressure sensors, wherein the controller is configured to generate an alert in response to receiving a signal from the pressure sensor indicating that the blood pressure is greater than a maximum blood pressure or is less than a minimum blood pressure.

In some implementations, the system includes one or more pressure sensors for measuring a blood pressure in the radial artery or a vessel upstream of the radial artery; and a controller configured to receive a signal from the one or more pressure sensors.

In some implementations, the controller is configured to generate an alert in response to receiving a signal from the pressure sensor indicating a blood pressure or change in blood pressure that exceeds a threshold or is below a threshold.

In some implementations, the controller is configured to control the pump to adjust the blood draw flow rate in response to receiving a signal from the pressure sensor indicating a blood pressure or change in blood pressure that exceeds a threshold or is below a threshold.

In some implementations, the vessel upstream of the radial artery is the brachial, axillary, or subclavian artery.

In some implementations, drawing blood without collapsing the artery or vessel to a degree that would substantially impede drawing blood comprises preventing a collapse that would result in more than 5-10% reduction in cross-sectional area of an artery or vessel.

In some implementations, the catheter comprises a coiled lumen configured to prop open a vessel of the patient. In some implementations, the catheter comprises a straight lumen. In some implementations, the catheter comprises a coiled lumen configured to prop open a vessel of the patient. In some implementations, the catheter comprises a straight configuration upon insertion and assumes a coiled configuration inside the artery or vessel.

In some implementations, the catheter comprises a straight lumen.

In a general aspect, a system for delivering gas-enriched blood within a vasculature of a patient includes: a gas-enrichment system configured to generate a gas-enriched liquid; a mixing chamber configured to generate a gas-enriched blood by mixing blood from the patient with a gas-enriched liquid received from the gas-enrichment chamber; a first catheter coupled to the mixing chamber, the first catheter comprising one or more lumens configured to receive the gas-enriched blood from the mixing chamber, the first catheter configured to be inserted into a first radial artery of a patient and deliver the gas-enriched blood to the patient; a second catheter configured to be inserted into a second radial artery of the patient, the second catheter comprising one or more lumens configured to draw blood from the radial artery of the patient or from a vessel upstream of the radial artery and to send the withdrawn blood to the mixing chamber; and a pump configured to cause the second catheter to draw the blood and configured to cause the first catheter to deliver the gas-enriched blood.

In some implementations, the blood flow rate is a predetermined blood flow rate of 10-500 mL/min, 30-300 mL/min, or 50 -150 mL/min.

In some implementations, second catheter is configured to be advanced to the vessel upstream of the radial artery and to draw the blood from the vessel upstream of the radial artery at a predetermine flow rate without collapsing the artery or vessel to a degree that would substantially impede drawing blood, wherein the predetermine blood flow rate is 50 -150 mL/min.

In some implementations, an inner diameter and length of the catheter are configured to support a predetermined blood flow rate of 50 -100 mL/min while preventing a pressure drop greater than a threshold pressure drop causing pump cavitation.

In some implementations, the inner diameter is 6-7 French, the length is 10-100 centimeters and the pressure drop is from 0 mmHG to negative 100 mmHG

In some implementations, the one or more sensors are configured to measure one or more blood parameters, and wherein operation of the first catheter, the second catheter, or both the first catheter and the second catheter are controlled based on the measured one or more parameters.

In some implementations, the one or more sensors comprises a blood pressure sensor, pO₂ sensor, SO₂ sensor, or a flow rate sensor.

In some implementations, the system includes a control system configured to control operation of the first catheter, the second catheter, or both the first catheter and the second catheter based on one or more signals representing the measured one or more parameters.

In some implementations, the control system is configured to: receive one or more signals representative of the measured one or more parameters from the one or more sensors; and based on the one or more signals, adjust a speed of the pump to alter a flow rate or flow pressure of the blood.

In some implementations, the one or more sensors comprises a pressure measuring device operable to measure blood pressure of the withdrawn blood.

In some implementations, the pressure measuring device is a pressure tube inserted through a communicating lumen in the second catheter, which communicating lumen is in fluid communication with the second radial artery of the patient, the pressure tube proximally connected to a pressure monitor.

In some implementations, the pressure measuring device is configured to measure blood pressure of the withdrawn blood in a draw line connecting the second catheter to the pump.

In some implementations, the pressure measuring device is a manometer mounted at a proximal end of the second catheter.

In some implementations, the system includes a control system configured to: receive the one or more signals representative of the measured one or more parameters from the one or more sensors, wherein the one or more parameters include pO₂ in the blood of the patient; and based on the one or more signals of the measured pO₂, adjust a concentration of oxygen in the gas-enriched liquid.

In some implementations, adjusting the concentration of oxygen in the gas-enriched liquid includes: increasing the concentration of oxygen in an initial control phase; and gradually reducing concentration of oxygen in a subsequent control phase until the pO₂ is within a pO₂ target range.

In some implementations, the one or more parameters comprise one or more of a blood pressure, pO₂, SO₂, and a flow rate of the blood of the patient.

In some implementations, an IR sensor is used to measure SO₂ in the blood of the patient.

In some implementations, the gas-enriched liquid comprises a supersaturated oxygen liquid.

In some implementations, the supersaturated oxygen liquid has an O₂ concentration of 0.1-6 ml O₂/ml liquid (STP). In some implementations, the second catheter is configured for insertion into a subclavian artery of the patient through the radial artery.

In some implementations, the one or more lumens of the second catheter comprise a braided pattern.

In some implementations, the braided pattern comprises a rectangular cross section.

In some implementations, the one or more lumens of the second catheter each comprise a wall thickness between 0.005 inches to 0.015 inches, the wall thickness preventing kinking of the one or more lumens of the second catheter.

In some implementations, the one or more lumens of the second catheter comprise an atraumatic tip.

In some implementations, the system includes a first sheath configured for inserting into the first radial artery of the patient, the first sheath configured to support the first catheter in the first radial artery.

In some implementations, the first sheath comprises a braided wire and a plastic liner over the braided wire. In some implementations, the first sheath is between 50-100 centimeters in length.

In some implementations, the system includes a second sheath configured for inserting into the second radial artery of the patient, the second sheath configured to support the second catheter in the second radial artery of the patient.

In some implementations, the second sheath comprises a braided wire and a plastic liner over the braided wire. In some implementations, the second sheath is between 50-100 centimeters in length. In some implementations, the second catheter comprises a distal band on a distal portion of the second catheter, the distal band configured to align with a predetermined location in the second radial artery of the patient.

In some implementations, the system includes one or more clamps or valves for controlling draw of the blood from the patient and delivery of blood to the patient.

In some implementations, the system includes a bubble trap configured to remove air from the gas-enriched blood prior to delivery of the blood to the patient.

In some implementations, the second catheter is advanced to a vessel upstream of the radial artery and blood is drawn from the vessel upstream of the radial artery.

In some implementations, the system includes one or more flow sensors for measuring a draw rate; and a controller configured to receive a signal from the one or more flow sensors, wherein the controller is configured to generate an alert in response to receiving a signal from the flow sensor indicating that the draw rate is greater than a maximum draw rate or is less than a minimum draw rate.

In some implementations, the system includes one or more pressure sensors for measuring blood pressure in the radial artery; and a controller configured to receive a signal from the one or more pressure sensors, wherein the controller is configured to generate an alert in response to receiving a signal from the pressure sensor indicating that the blood pressure is greater than a maximum blood pressure or is less than a minimum blood pressure.

In some implementations, the system includes one or more pressure sensors for measuring a blood pressure in the radial artery or a vessel upstream of the radial artery; and a controller configured to receive a signal from the one or more pressure sensors.

In some implementations, the controller is configured to generate an alert in response to receiving a signal from the pressure sensor indicating a blood pressure or change in blood pressure that exceeds a threshold or is below a threshold.

In some implementations, the controller is configured to control the pump to adjust the blood draw flow rate in response to receiving a signal from the pressure sensor indicating a blood pressure or change in blood pressure that exceeds a threshold or is below a threshold.

In some implementations, the vessel upstream of the radial artery is the brachial, axillary, or subclavian artery.

In some implementations, the second catheter comprises a coiled lumen configured to prop open a vessel of the patient.

In some implementations, the second catheter comprises a straight lumen.

In some implementations, drawing blood without collapsing the artery or vessel to a degree that would substantially impede drawing blood comprises preventing a collapse that would result in more than 5-10% reduction in cross-sectional area of an artery or vessel wherein the blood flow rate is a predetermined blood flow rate of 10-500 mL/min, 30-300 mL/min, or 50-150 mL/min.

In some implementations, drawing blood without collapsing the artery or vessel to a degree that would substantially impede drawing blood comprises preventing a reduction of blood flow over a threshold percentage of 10-15%.

In some implementations, the blood flow rate is a predetermined blood flow rate of 10-500 mL/min and blood flow in an artery is not reduced by more than a predefined threshold percentage of 10-15%.

In some implementations, a system is configured for drawing blood without collapsing the artery or vessel to a degree that would substantially impede drawing blood comprises preventing a reduction of blood flow over a threshold percentage of 10-15%.

In some implementations, a system is configured for drawing blood without collapsing the artery or vessel to a degree that would substantially impede drawing blood comprises preventing a collapse that would result in more than 5-10% reduction in cross-sectional area of an artery or vessel wherein the blood flow rate is a predetermined blood flow rate of 10-500 mL/min, 30-300 mL/min, or 50-150 mL/min

In general, an implementation described with respect to one aspect may be provided in combination with another aspect. The details of one or more embodiments are set forth in the accompanying drawings and the description. Other features and advantages will be apparent from the description and drawings, and from the claims.

### BRIEF DESCRIPTION OF THE DRAWINGS

FIG. 1 is a diagram of an example gas-enrichment system for delivering gas-enriched blood within the vasculature of a patient.

FIG. 2A is a diagram of example catheters in the vasculature in the patient for bi-radial access of the vasculature for each of the draw line and the return line of the gas-enrichment system of FIG. 1.

FIG. 2B is a diagram of an example draw catheter in the vasculature in the patient for radial access of the vasculature for the draw line of the gas-enrichment system of FIG. 1, the draw catheter being advanced into the subclavian artery.

FIG. 2C is a diagram of an example catheter in the vasculature in the patient for radial access of the vasculature for the draw line of the gas-enrichment system of FIG. 1, including a sheath.

FIG. 2D is a diagram of an example catheter in the vasculature in the patient for radial access of the vasculature for both draw and return lines of the gas-enrichment system of FIG. 1.

FIG. 2E is a diagram of example catheters in the vasculature in the patient for bi-radial access of the vasculature for each of the draw line and the return line of the gas-enrichment system of FIG. 1.

FIGS. 3A-3B are diagrams of an example draw catheter for radial access of the vasculature in the patient.

FIG. 4 is a diagram of a portion of the system of FIG. 1-3 including a cartridge.

FIG. 5 shows a perspective view of the system of FIGS. 1-4.

FIG. 6 shows a block diagram of a process for gas-enrichment therapy.

FIG. 7 shows an example computer system.

The drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

### DETAILED DESCRIPTION

The following disclosure describes systems and methods related to, and example embodiments of, gas enrichment therapy or supersaturated oxygen or gas therapy systems, methods and components. The systems permit gas-enrichment therapy, e.g., supersaturated oxygen (SSO₂) therapy to be provided to patients. The system may be controlled based on an analysis of one or more physiological parameters. SSO₂ therapy refers to minimally invasive procedures for enriching oxygen content of blood through catheter-facilitated infusion of supersaturated oxygen-enriched physiological fluid (e.g., blood) or infusion of supersaturated oxygen-enriched liquid, such as saline, directly into a patient's blood vessel. These procedures generally are aimed at treating a patient who has suffered an acute myocardial infarction (AMI), but can be used for other conditions, including, but not limited to, peripheral vascular disease as well. When delivering gas-enriched liquid (e.g., gas-enriched blood), a delivery system may moderate a draw rate from the vasculature of the patient to avoid collapsing an artery of the patient. The delivery systems described herein are configured to access the patient's vasculature via the radial artery such that blood is drawn from the radial artery or from other vessels via the radial artery of the patient without substantially collapsing the radial artery and/or the other vessels to a degree that would substantially impede drawing blood.

In certain implementations, substantially impeding blood flow within the vessel (such as from collapsing the blood vessel) can include the following. In an example, substantially impeding the blood flow of an artery (such as the radial artery) may include reducing blood flow over a threshold percentage, such as about 10-15% or about 15-25%. In some implementations, substantially impeding blood flow may include reducing blood flow (including blood draw) in the vessel by over 50%. In some implementations, substantially impeding blood flow may include reducing blood flow (including blood draw) in the vessel by at least about 10%. In some implementations, substantially impeding blood flow may include reducing blood flow (including blood draw) in the vessel such that arterial spasm is detectable. In some implementations, a pressure sensor, flow sensor, or a combination thereof is included on the catheters described herein or elsewhere in the blood circuit or a blood vessel of the patient. The sensor is configured to generate feedback from the blood circuit or the blood vessel to detect impeded blood flow. For example, a draw side pressure or flow sensor in the blood circuit or in the patient can be configured to detect an arterial spasm, which is indicative of impeded blood flow in the patient. An arterial spasm includes shrinking or constricting of the artery in which blood flow is irregular or reduced, and in which blood flow drops or blood pressure fluctuates to reduce blood flow (e.g., more than about a 10% reduction or change in blood flow volume or rate). In some implementations, drawing blood without collapsing the artery or vessel to a degree that would substantially impede drawing blood comprises preventing a collapse that would result in more than 5-10% reduction in cross-sectional area of an artery or vessel, wherein the blood flow rate is a predetermined blood flow rate of 10-500 mL/min, 30-300 mL/min, or 50-150 mL/min. In some implementations, drawing blood without collapsing the artery or vessel to a degree that would substantially impede drawing blood comprises preventing a reduction of blood flow over a threshold percentage of about 10-15%. In some implementations, the blood flow rate is a predetermined blood flow rate of 10-500 mL/min and blood flow in an artery is not reduced by more than a predefined threshold percentage of about 10-15%. In some implementations, a system is configured for drawing blood without collapsing the artery or vessel to a degree that would substantially impede drawing blood comprises preventing a reduction of blood flow over a threshold percentage of about 10-15%.

As described previously, the delivery systems described herein may be configured to deliver gas-enriched liquid (e.g., gas-enriched blood) to the radial or other vasculature of the patient. The blood circuit may be a biradial blood circuit in which blood is drawn from and returned to the radial vasculature (e.g., radial arteries) and/or other vessels upstream of the radial artery, e.g., brachial, axillary, or subclavian arteries of the patient. The delivery system is configured to allow blood draw from the radial artery or from other vessels via the radial artery in a precise and controlled manner to avoid collapse of the radial artery and/or the other vessels.

In some implementations, a draw line is inserted into a radial vasculature of the patient and advanced to a subclavian artery. The subclavian arteries include a pair of large arteries in the thorax that supply blood to the thorax itself and the head, neck, shoulder, and arms of the patient. Depending on the side of the body, the subclavian artery can have two origins the aortic arch on the left and the brachiocephalic trunk on the right. The draw line catheter can be advanced to or inserted into the subclavian artery to allow for a larger quantity of blood to be drawn from the patient at a faster draw rate (e.g., without collapsing the artery).

In certain implementations, the delivery system described in this document is configured to perform measurements of one or more physiological parameters (such as blood flow or blood pressure or both) to determine how to adjust control of blood draw and/or delivery of gas-enriched blood to the patient. Controlling gas-enrichment therapy may refer to a process of adjusting the delivery of gas-enriched blood or liquid either to increase the amount delivered or decrease the amount delivered over a period of time (e.g., several seconds to a several minutes) or increase or decrease the time of delivery or to stop or start delivery of the gas enriched blood or liquid, or to increase or decrease the amount of gas dissolved in liquid, which is then mixed with blood, in a precisely controlled way. For example, controlling delivery of the gas enriched blood or liquid can include titrating the delivery of the gas-enriched blood or liquid. The delivery system may be configured for measuring the physiological parameters during delivery of the gas-enriched blood to control a first catheter attached to the draw line and a second catheter attached the return line.

In certain implementations, the delivery system is configured for real-time control of blood draw or the delivery of the gas-enriched blood (e.g., real-time control loop) for controlling the draw rate (e.g., pump speed) or the delivery of the gas-enriched blood or liquid to the patient using radial access to the patient. Real-time in this context refers to an instant or nearly instant generation of a control signal in response to receiving data from one or more sensors in communication with a controller of the delivery system. The control signal is generated with minimal delay, allowing for processing latencies and/or communication latencies inherent to measuring the physiological parameter values and processing the measured data. Real-time therefore refers to processing the measured parameter values as the data are received at the processor rather than storing the measured values for use in processing at a later time. For example, the delivery system can continually update a value in a sensor buffer representing the most recent measurement of the physiological parameter that is available to the controller for processing.

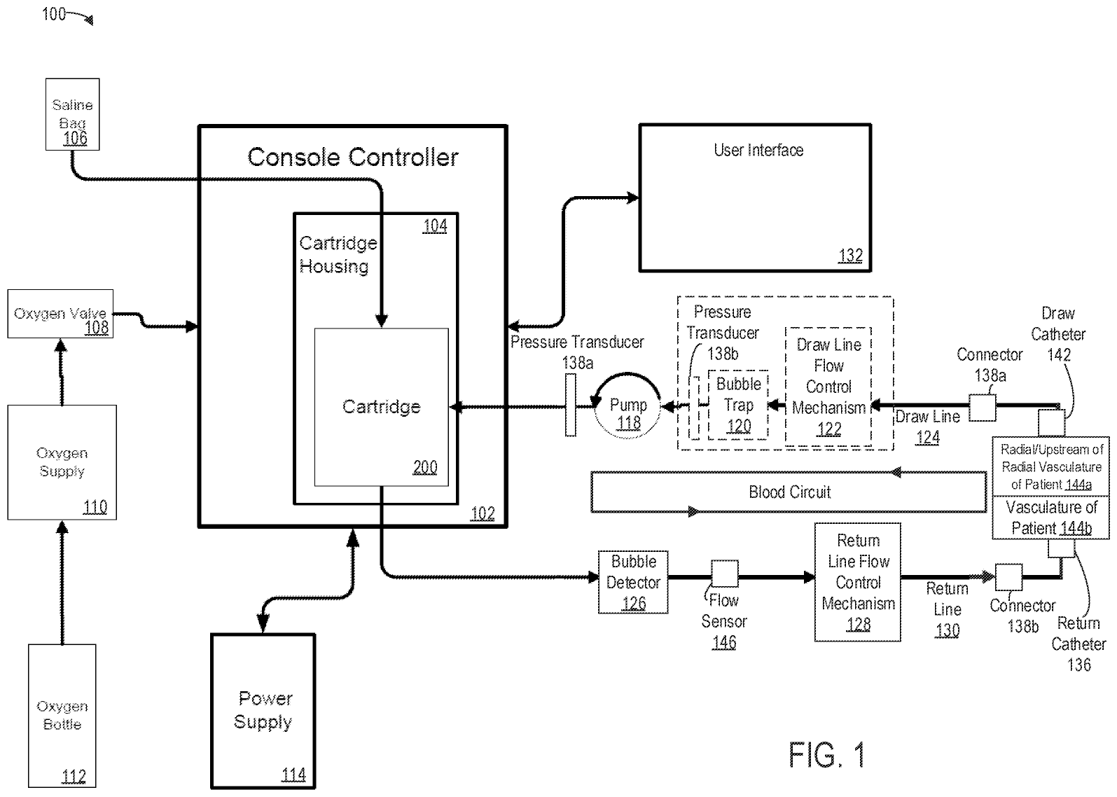

FIG. 1 is a diagram of an example gas-enrichment and delivery system 100 for delivering gas-enriched blood within the vasculature of a patient. The delivery system 100 can enable enrichment of a bodily fluid (e.g., blood) with a dissolved gas or gas-enriched liquid. As an example, the delivery system 100 creates a gas-enriched blood by enriching a patient's blood with a gas-enriched liquid, e.g., oxygen enriched liquid, in an extracorporeal gas-enrichment and control system including a controller 102 and a cartridge 200. Gas-enriched blood, e.g., oxygen enriched blood or supersaturated oxygen (SSO₂) enriched blood, is delivered to a patient 144, thereby increasing oxygen in the blood of the patient and diffusion of oxygen into tissue to treat ischemic (oxygen-deprived) tissue, e.g., in patients who have suffered a myocardial infarction.

In certain implementations, oxygen enriched liquid or solution, e.g., supersaturated oxygen liquid or solution, may include liquid having a dissolved O₂ concentration of 0.1 ml O2/ml liquid (STP) or greater or 0.1-6 ml O2/ml liquid (STP) or 0.2-3 ml O2/ml liquid (STP) (e.g., without clinically significant gas emboli). When such supersaturated oxygen liquid or solution is mixed with blood, the resulting blood may be referred to as supersaturated oxygen enriched blood. In certain implementations, the system 100 may deliver an infusion of supersaturated oxygen enriched blood having an elevated pO₂ in a target range of 400 mmHg or greater or 600-1500 mmHg or 760-1200 mmHg or around 1000 mmHg.

In one example, supersaturated oxygen enriched blood may have a pO2 of 760-1500 mmHg when a source blood delivered to the gas enrichment system for mixing with a supersaturated oxygen liquid or solution has a minimum pO₂ of 80 mmHg, the blood flow rate is 50-150 ml/min, the SSO2 saline flow rate is 2-5 ml/min and the dissolved O₂ concentration in saline is 0.2-3 ml O2/ml saline (STP).

In another example, where the source blood is below 80 mmHg, the treatment objective may be to boost the blood pO2 to above 80 mmHg, so the system 100 may deliver an infusion of supersaturated oxygen enriched blood having a pO2 level of 80 mmHg or greater or 80-760 mmHg.

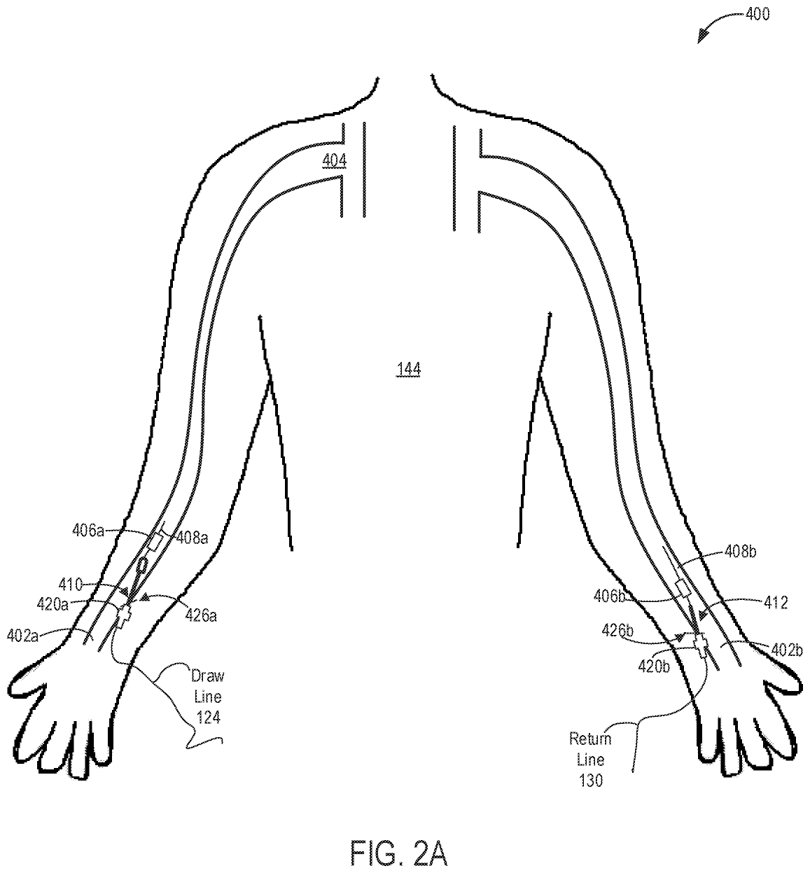

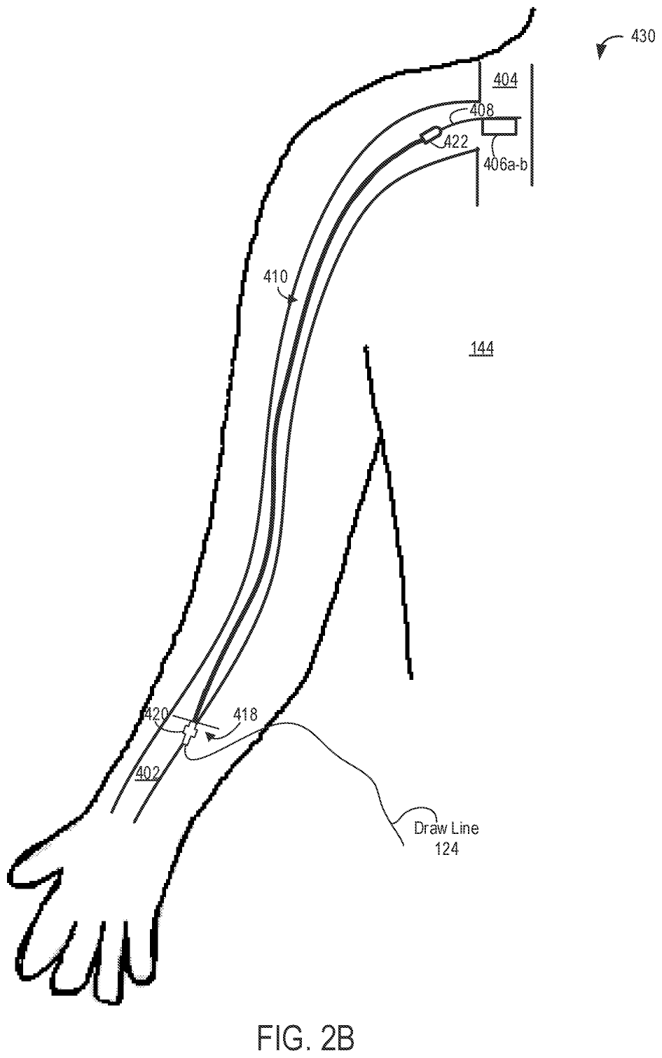

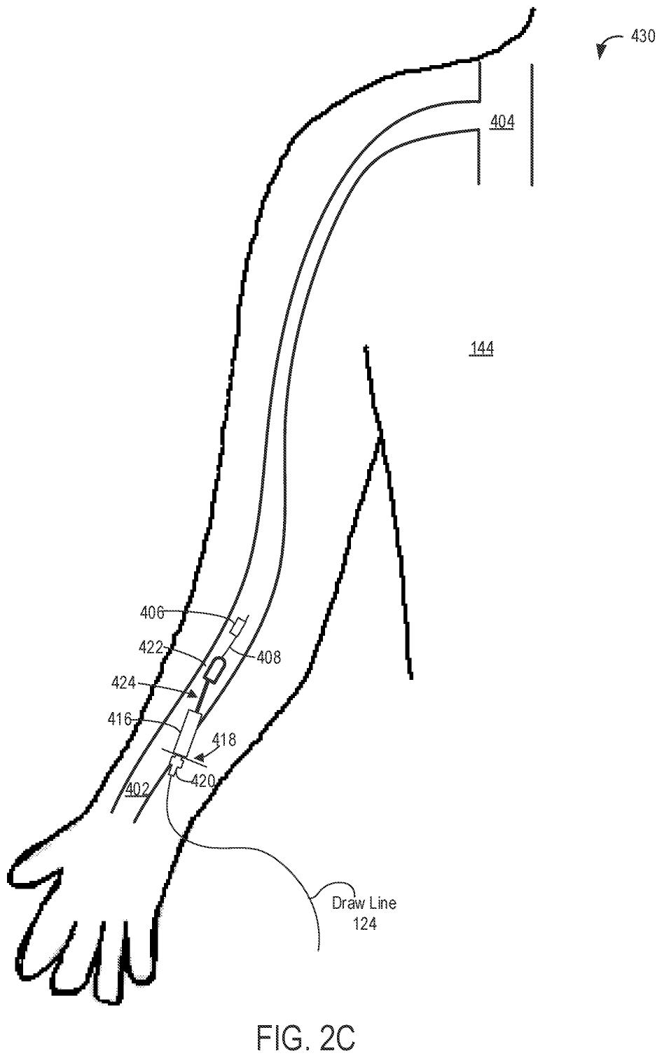

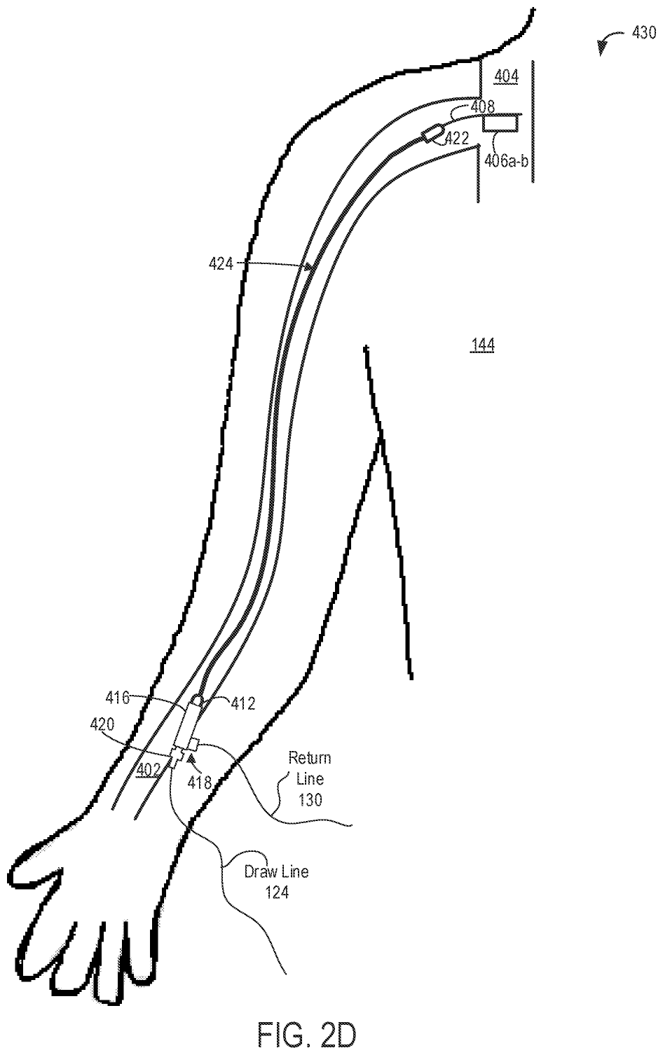

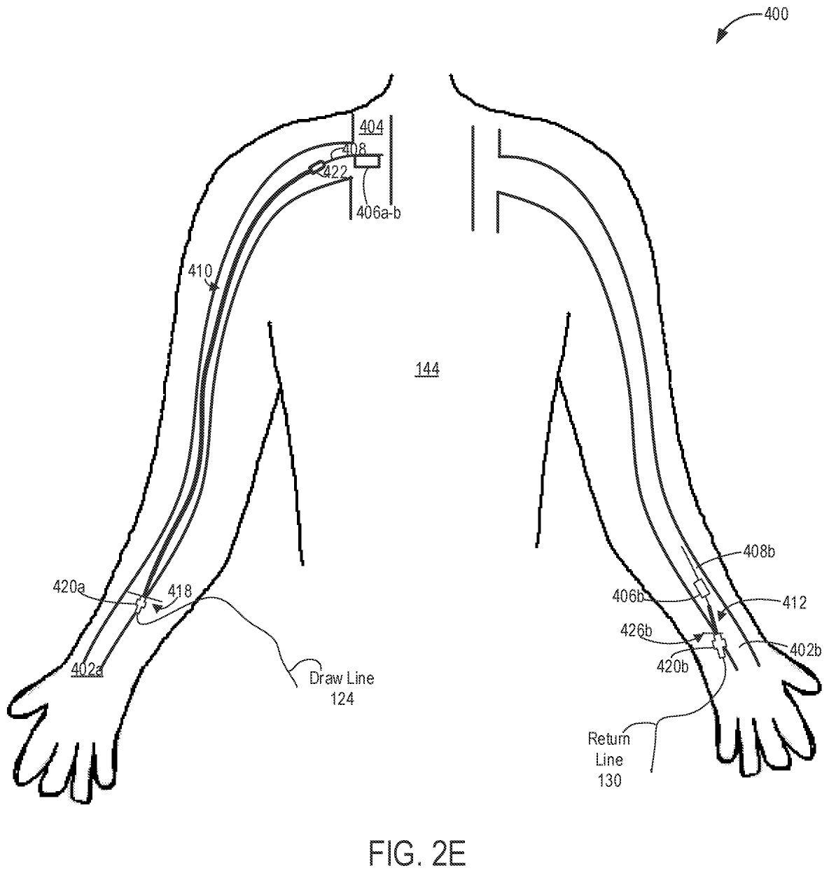

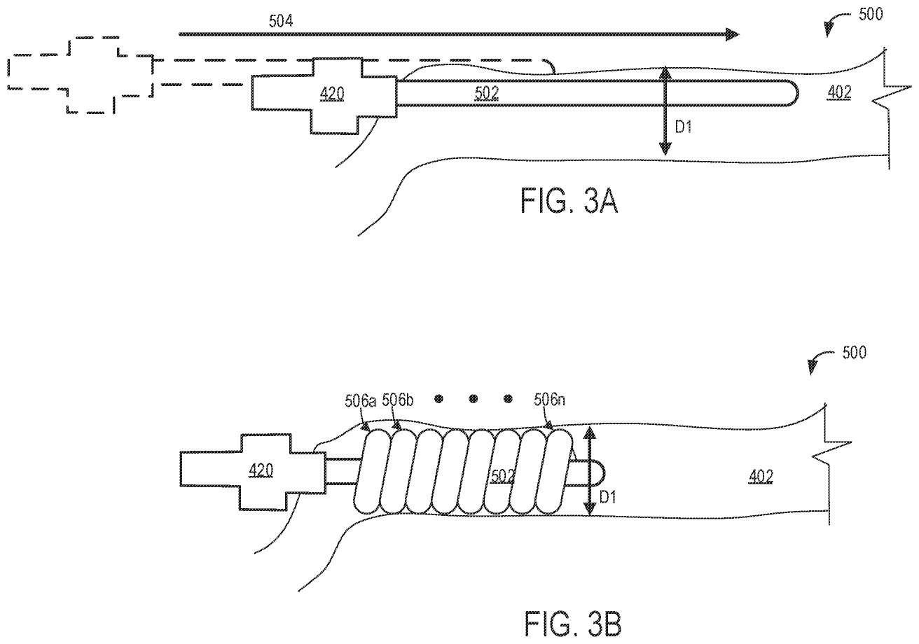

In certain implementations, the delivery system 100 is configured to perform real-time or near real-time measurements of the blood flow rate or pressure (e.g., the change in pressure over time) in the radial artery and/or other vessel upstream of the radial artery, e.g., brachial, axillary, or subclavian artery, and use that feedback regarding the blood flow rate or pressure in the radial artery and/or other vessel upstream of the radial artery, e.g., brachial, axillary, or subclavian artery, to adjust a draw rate of blood from the patient (e.g., by adjusting pump speed) to avoid collapse of the radial artery and/or other vessel upstream of the radial artery, e.g., brachial, axillary, or subclavian artery, of the patient to a degree that would substantially impede drawing blood. The delivery system 100 may include one or more sensors for measuring blood flow in the patient, as described in relation to FIGS. 2A-3.

In certain implementations, the delivery system 100 may include one or more pressure sensors for measuring a blood pressure in the radial artery, a vessel upstream of the radial artery and/or in the draw line. The delivery system may include a controller configured to receive a signal from the one or more pressure sensors. The controller may be configured to generate an alert in response to receiving a signal from the pressure sensor indicating a blood pressure or change in blood pressure that exceeds a threshold or is below a threshold. The controller may be configured to control the pump to adjust the blood draw flow rate in response to receiving a signal from the pressure sensor indicating a blood pressure or change in blood pressure that exceeds a threshold or is below a threshold.

The delivery system 100 is configured for controlling gas-enrichment therapy in a patient by enriching a liquid with gas to form a gas-enriched liquid and to mix the gas-enriched liquid with blood to form gas-enriched blood. A pump 118, subsequently described in further detail, is configured to pump blood to and from the gas-enrichment system to and from the patient through a plurality of fluid conduits fluidly coupled to the gas-enrichment system.

In certain implementations, at least one sensor (e.g., described in relation to FIG. 1B) is configured to measure one or more physiological parameters. A controller 102 comprising a processor, a memory, and associated circuitry is communicatively coupled to the at least one sensor. The processor is configured to receive one or more signals corresponding to a measured value of the one or more physiological parameters (e.g., blood pressure or flow rate) from the at least one sensor. The controller 102 is configured to control, based on the values of blood pressure or blood flow rate in the vasculature of the patient, an alert or control signal for sending to the pump 118 for adjusting delivery or draw of blood from the patient.

The blood circuit includes the blood mixing chamber of the cartridge that receives blood from the patient 144 and where enrichment of the blood with gas-enriched liquid occurs. The blood circuit may also include an air trap 120 or bubble trap chamber. The blood mixing chamber and/or bubble trap 120 may include one or more level sensors 160, e.g., ultrasound sensors, for detecting the presence or absence of liquid in the respective chamber or trap. These sensors 160 may send signals for controlling the flow control mechanisms depending on the presence or absence of liquid. The blood circuit also includes the tubing between and among these chambers. The blood circuit of the delivery system 100 is connected to an intravenous catheter 136 which is insertable into the vasculature of a patient 144 to complete the blood circuit. Blood is removed from the patient 144, drawn into the cartridge of the delivery system 100, mixed with gas-enriched liquid, e.g., oxygen-enriched saline, and returned to the patient. The chambers of the blood circuit may include one or more chambers of the cartridge 200, the bubble trap 120. A bubble detector 126 may also be provided for detecting air bubbles in the blood circuit.

In certain implementations, the delivery system 100 may include a console controller 102 cartridge housing 104, a user interface 132, a pump 118, a power supply 114, and an oxygen valve 108 and associated oxygen supply connector 110. The delivery system is configured to connect to several consumable items that are used as a part of the delivery system 100, including an oxygen bottle 112, fluid source 106 (or saline bag 106), a cartridge 200 and the catheter 136.

The delivery system 100 further includes a draw line 124 for drawing blood from a draw catheter 142 through connector 138a. As described herein and specifically in relation to FIGS. 2A-2D, the draw catheter is inserted in the vasculature of the patient 144 through the radial artery. The draw line 124 may include a bubble trap chamber 120 and is configured to interface with a pump 118 and may be configured to interface with a first flow control mechanism, e.g., a draw line flow control mechanism 122 of the delivery system 100. Pressure transducers 138a-b may be located on either side of the pump 118 to measure pressure of blood flowing through the blood circuit, such as through the draw line 124, through the return line 130, or through each of the draw line and the return line.

The draw line 124 is connected to the draw catheter 142. For example, the draw catheter can be a single-use consumable device that is used once before being discarded. The draw catheter 136 includes a lumen for drawing blood from the patient 144. The draw line 124 may be connected (e.g., by connector 138a) to the catheter 142 to draw blood from the patient 144. In some implementations, the draw catheter 142, which includes a lumen for drawing blood from the patient may be inserted through a sheath 142 positioned in the patient's vasculature. In this example, the draw line 124 is connected to the catheter 142.

The draw catheter 142 is configured to draw blood from a radial artery and/or from a vessel upstream of the radial artery, e.g., the brachial, axillary, or subclavian artery, of the patient 144. Generally, the delivery system 100 is configured to draw at least 100 cubic centimeters (cc's) per minute or 100 ml/min of flow rate from the patient. The delivery system 100 draw pressure may be maintained within a range of 50 to 100 mmHg. The blood draw line pressure is maintained in the draw line 124 to avoid a pressure drop that is greater than a threshold pressure drop value to avoid cavitation by the blood pump 118 in which air bubbles or air cavities are formed in the blood circuit. A length of the draw catheter 142 is short enough to enable the draw pressure in the draw line 124 from the radial artery and/or from a vessel upstream of the radial artery, e.g., the brachial, axillary, or subclavian artery to be maintained above the threshold value to prevent the radial artery and/or a vessel upstream of the radial artery, e.g., the brachial, axillary, or subclavian artery from collapsing due to low pressure in the radial artery or other vessel. In some implementations, the catheter may be sized and configured, such that the pressure drop from the catheter to the pump does not exceed a threshold value in order to prevent cavitation. In some implementations, the draw catheter 142 is located in the brachial or subclavian artery, which are of a larger diameter than the radial artery. In some implementations, the draw catheter 142 is located in the radial artery and has a length or other mechanism to prevent artery collapse that would to a degree substantially impede drawing blood or blood flow.

The delivery system 100 includes a return line 130 for returning gas-enriched blood to the catheter 136 in the patient 144. The return line 130 may be connected through a bubble detector 126, and connected to the catheter via a connector 138b. The return line may be configured to interface with a second flow control mechanism, e.g., a return line flow control mechanism 128. A draw clamp or valve can be used to perform the functions of the draw flow control mechanism 122, and a return clamp or valve can be used to perform the functions of the return flow control mechanism 128. In some implementations, another mechanism for controlling or regulating flow of the blood in the blood circuit (e.g., to prevent blood flow and/or flow of room air or air bubbles) can be used to perform the functions of the draw flow control mechanism 122 or return flow control mechanism 128.

A return catheter 136 is connectable to the delivery system 100. For example, the return catheter 136 can be a single-use consumable device that is used once before being discarded. The return catheter 136 includes a lumen for delivering gas-enriched blood to the patient 144. The return line 130 may be connected (e.g., by connector 138b) to the catheter 136 to return blood to the patient 144. In some implementations, the return catheter 136, which includes a lumen for delivery of the gas-enriched blood to the patient may be inserted through a sheath 142 positioned in the patient's vasculature. In this example, the return line 130 is connected to the catheter 136. Here, the vasculature of the patient 144 is divided into two portions. For the draw catheter 142, the vasculature is the radial vasculature and/or other vasculature upstream of the radial artery, e.g., the brachial, axillary, or subclavian artery 144a of the patient. For the return catheter 136, the vasculature includes radial or other vasculature 144b, such as femoral vasculature.

In certain implementations, the blood circuit the draw line 124 may be connected (e.g., by connector 138a) to a sheath 142 inserted into the patient 144 for drawing blood from the patient 144. The sheath 142 includes a lumen for drawing blood from the patient 144, and the draw line 124 is connected to the sheath 142.

In certain implementations, the catheter 142 may be used for drawing blood from the patient at a location different than the location of the return catheter 136. The delivery system 100 uses two separate catheters 136, 142 to control each of delivery/return and draw of the blood from the patient 144. In some implementations and as subsequently described, the draw catheter 142 can be shaped to assist in preventing collapse or substantial collapse of the radial artery and/or a vessel upstream of the radial artery, e.g., the brachial, axillary, or subclavian artery of the patient to a degree that would impede or substantially impede drawing blood. In some implementations, the draw catheter 142 is inserted into the radial artery. In some implementations, the draw catheter 142 is inserted into the subclavian artery of the patient through the radial artery. When the subclavian artery is used, the draw catheter 142 can be larger and configured for greater draw of the blood from the patient 144 into the blood circuit.

In other implementations, the draw or return catheter 142/136 may include a second lumen for drawing blood from the patient 144 so that a sheath is not used, and so the catheter 142/136 is configured for both returning and drawing blood from the patient 144. In this example, the draw line 124 and the return line 130 are connected to the catheter 136. The delivery system 100 is configured for use with different types of catheters. In another example, the sheath 142 includes a first lumen for connecting to the draw line 124 for, drawing blood from the patient 144 and a second lumen for connecting to the return line 130 for returning blood to the patient.

Turning to FIGS. 2A-2D, radial placement of the draw and return catheters 136, 142 are shown in the patient vasculature 400. The catheters 136, 142 are configured to operate for gas-enrichment therapy, e.g., SSO₂ treatment, as described in relation to the delivery system 100 of FIG. 1.

FIG. 2A shows an example vasculature 400 of the patient 144 of FIG. 1. The draw line 124 is connected (e.g., via connector 138a) to a draw catheter 410 (e.g., draw catheter 142 of FIG. 1). The draw catheter 410 is positioned in a radial artery 402a of the patient 144. The draw catheter 410 is sized to enable draw of a particular amount of blood (e.g., 100 mL/min) while enabling the blood pressure or change in blood pressure in the radial artery 402a to be maintained above or below a threshold value. While FIG. 2A shows an example of a catheter positioned in the radial artery for blood draw from the radial artery, in other examples a catheter may be sized and configured for advancement (via radial artery access) into other vessels upstream of the radial artery, e.g., the brachial, axillary, or subclavian artery, to allow for draw of a particular amount of blood (e.g., 100 mL/min) from those arteries while allowing the blood pressure or change in blood pressure in those arteries to be maintained relative to a threshold value.

In certain implementations, the draw catheter 410 may include one or more sensors 406a. For example, the draw catheter may include a wire 408a that extends from a distal end of the catheter 410 into the radial artery 402a or vessel upstream of the radial artery of the patient. The wire 408a may support a sensor module 406a. The sensor module 406a can include one or more sensors that are instances of a same type of sensor or different types of sensors. In an example, each of the sensors of module 406a includes a pressure sensor configured to measure blood pressure or a change in blood pressure in the radial artery 402a of the patient. Two pressure sensors can provide a pressure differential (Δp) value for a region of the radial artery 402a. The wire 408a is inserted into the radial artery 402a and can record a blood pressure in the radial artery 402a. This can ensure that a minimum blood pressure is maintained in the radial artery 402a to prevent collapse of the radial artery during blood draw from the radial artery. In some implementations, the sensors module 406a includes flow sensors configured to measure the flow of blood directly in the radial artery 408a. The flow sensors can include temperature, electromagnetic, mechanical, or ultrasonic flow sensors. For example, a pressure sensor on the distal end of the wire can act as a distal thermistor, while a pressure sensor on the proximal shaft of the wire serves as a proximal thermistor. Accordingly, a mean transit time (Tₘₙ) of room-temperature saline injected into a coronary artery can be determined from a thermodilution curve. Using the thermodilution technique, a correlation between the inverse of Tₘₙ (1/_{Tmn}) and absolute coronary flow is shown. Absolute coronary flow≈1/Tₘₙ. In some implementations, a pressure sensor may measure a blood pressure change over time, e.g., a blood pressure drop in the radial artery or draw catheter, and provide feedback to ensure that the pressure drop does not exceed a threshold value, which would result in a vessel collapsing to a degree that would substantially impede drawing blood. In certain implementations, where the catheter is inserted into other vessels upstream of the radial artery, e.g., the brachial, axillary, or subclavian artery, the sensors would be positioned in the respective vessel and configured to measure blood pressure or blood pressure change over time or a pressure differential for a region of the vessel or blood flow rate in said vessel or draw catheter to prevent vessel collapse or cavitation.

In certain implementations, pressure and or flow may be measured and provide feedback in order to prevent or reduce vessel collapse that would substantially impede blood flow or drawing of blood. For example, in certain arteries, the system may prevent a collapse resulting in more than 5-10% reduction in cross-sectional area of an artery or vessel. In some implementations, the sensors of the sensor module 406a can detect arterial spasms, which can be indicative of impeded blood flow. Arterial spasms include shrinking or constricting of the artery in which blood flow is irregular or reduced, and in which blood flow drops or blood pressure fluctuates to reduce blood flow (e.g., by more than 10%). In certain implementations, the senor module may include a flow and/or pressure sensor. In some implementations, the one or more sensors of the sensor modules 406a-b can be positioned in either or both of the draw line or the return line. In some implementations, the one or more sensors of the sensor modules 406a-b can be positioned in the vasculature of the patient, as shown in FIG. 2E. In some implementations, the one or more sensors of the sensor modules 406a-b can be positioned in a combination of locations, such as in the vasculature of the patient, in either or both of the draw line and return line, and in the console of the delivery system 100.

In some implementations, the wire 408a is positioned on or near a distal end of the draw catheter 410. In some implementations, the module 406a is positioned directly on the body of the catheter 410. The wire 408a can extend through a lumen of the catheter 410 out of the distal end of the catheter. In some implementations, the wire 408a extends along the catheter 410 shaft on an exterior of a catheter lumen. In some implementations, the wire 408a is positioned on a separate probe that is not directly attached to the draw catheter 410. In some implementations, a second, separate catheter is used to support the one or more sensors.

Similarly, a return catheter 412 (e.g., similar to return catheter 136 of FIG. 1) is positioned in a radial artery 402b of the patient 144 and is connected to the blood circuit (e.g., via connector 138b. In the example shown in FIG. 2A, the draw catheter 410 is positioned in a first radial artery 402a of the patient, and the return catheter 412 is positioned in a second radial artery 402b of the patient in a different arm of the patient. In some implementations, the draw catheter 410 and return catheter 412 are positioned in the same radial artery in the same arm. In certain implementations, the draw and return catheters may be inserted into the vasculature via a first and second sheath.

In certain implementations, the return catheter may include one or more sensors. For example, the return catheter 412 may include a wire 408b that extends from a distal end of the catheter 412 into the radial artery 402b or vessel upstream of the radial artery of the patient. The wire 408b supports sensor module 406b. The sensor module 406b can include one or more sensors that are instances of a same type of sensor or different types of sensors. In an example, each of the sensors of module 406b includes a pressure sensor configured to measure blood pressure in the radial artery 402b of the patient. The two pressure sensors can provide a pressure differential (Δp) value for a region of the radial artery 402b or a change in pressure over time. The wire 408b is inserted into the radial artery 402b and can record a blood pressure in the radial artery 402b. In some implementations, the sensors module 406b includes flow sensors configured to measure the flow of blood directly in the radial artery 408ba. The flow sensors can include temperature, electromagnetic, mechanical, or ultrasonic flow sensors. In certain implementations, where the catheter is inserted into other vessels upstream of the radial artery, e.g., the brachial, axillary, or subclavian artery, the sensors would be positioned in the respective vessel and measure pressure or flow therein.

In some implementations, the wire 408b is positioned on or near a distal end of the return catheter 412. In some implementations, the module 406b is positioned directly on the body of the catheter 412. The wire 408b can extend through a lumen of the catheter 412 out of the distal end of the catheter. In some implementations, the wire 408b extends along the catheter 412 shaft on an exterior of a catheter lumen. In some implementations, the wire 408b is positioned on a separate probe that is not directly attached to the return catheter 412. In some implementations, a second, separate catheter is used to support the one or more sensors.

The sensor modules 406a-b are each configured to send data to a controller (e.g., controller 102 of FIG. 1) which can display feedback on the user interface 132 responsive to receiving the data from the sensors. The feedback that is displayed on the user interface 132 can include a display of blood pressure values, blood flow rate values, a rate of delivery of the gas-enriched blood (e.g., a titration value), a pump speed and direction, and so forth. As subsequently described in additional detail, the feedback displayed by the user interface 132 can include a time series showing a sequence of measurements. In some implementations, the feedback can include a diagram or graph that is continually or intermittently updated as new data are acquired. For example, the pressure values can be graphed in relation to time.

The data from the sensor modules 406a-b is sent to the controller either automatically and directly or through action of a user. In some implementations, the data are sent from sensor modules 406a-b over the wire 408a-b to the controller 102, which is connected either directly to the wire or indirectly to the wire through a catheter hub (not shown).

Data generated from the sensors of the sensor modules 406a-b are used for controlling delivery of the gas-enriched blood to the vasculature of the patient. The delivery system 100 is configured to control blood draw and gas-enriched blood delivery based on the feedback of the sensors of sensor modules 406a-b. In some implementations, the delivery system 100 is configured to augment blood flow. In some implementations, the delivery system 100 is configured to increase a concentration of oxygen in the gas-enriched liquid/blood delivered to the patient. In some implementations, the delivery system 100 increases or decreases an amount of gas-enriched blood/liquid to the patient, either by increasing or decreasing the flow rate of blood draw or gas-enriched blood (e.g., SSO₂) delivery or by increasing or decreasing the duration of gas-enriched blood (e.g., SSO₂) delivery.

The control of the gas-enrichment therapy may be performed in real-time or near-real time. The delivery of the gas-enriched blood to the patient may not paused during measurement of the one or more physiological parameters. The measurement of the pressure or flow represents a contemporaneous status of the patient for the delivery of the gas-enriched blood to the patient. Generally, the real-time or near-real time comprises processing, by the controller, data received from the one or more sensors as soon as the data are available to the controller and generating the control signal based on the processing, as previously described.

The controller 102 is configured to control gas-enrichment (e.g., SSO2) delivery over time. The controller 102 receives one or more signals corresponding to a measured value of the one or more physiological parameters from the sensor modules 406a-b. The controller 102 can receive a series of measured values of the pressure values or flow values from the sensors over time. The series of measured values corresponds to a period of time during delivery of the gas-enriched blood to the patient. The controller 102 determines, based on the series of measured values, whether the value of the pressure or flow is increasing or decreasing over time. The controller 102 generates the control signal that is configured to increase or reduce the blood flow rate or amount of the gas-enriched blood delivered to the patient based on the time series of values.

In certain implementations, if the blood pressure values in a patient are increasing over time, the controller may provide an alert or reduce the pump speed, thereby reducing blood flow rate and blood pressure. For example, if the pressure values are decreasing over time, the controller may provide an alert or increase the pump speed, thereby increasing blood flow rate and blood pressure. If the pump speed and blood flow rate is modified, the SSO2 saline flow rate and/or the dissolved O2 concentration in saline may also need to be modified to achieve a desired pO2 in blood. For example, supersaturated oxygen enriched blood may have a pO2 of 760-1500 mmHg when a source blood delivered to the gas enrichment system for mixing with a supersaturated oxygen liquid or solution has a minimum pO₂ of 80 mmHg, the blood flow rate is 50-150 ml/min, the SSO2 saline flow rate is 2-5 ml/min and the dissolved O₂ concentration in saline is 0.2-3 ml O2/ml saline (STP), and such parameters may be modified depending on changes to one or more of the other parameters. For example, if the blood flow rate is reduced, either the SSO2 saline flow rate or dissolved O₂ concentration in saline could be reduced to maintain desired pO2 levels. Reducing oxygen pressure may reduce both SSO2 saline flow rate and dissolved O₂ concentration in saline.

Generally, the controller 102 controls draw of the blood from the draw catheter 410 to maintain a pressure in the blood circuit without dropping pressure in the radial artery, or other vessels upstream of the radial artery, e.g., the brachial, axillary, or subclavian artery 402a, too low such that the blood vessel would collapse to a degree that would impede or substantially impede blood flow or drawing blood. A catheter 410 length may enable drawing blood from a region, e.g., at 100 mL/min draw rate, without collapse to a degree that would substantially impede blood flow or drawing blood (e.g., from the subclavian artery, as shown in relation to FIG. 2B).

In some implementations, a low pressure monitor, pressure sensor, is used at a connection of the draw line 124 to the draw catheter 410 or sheath (see FIG. 2C) or at the pump or at the distal end the catheter. The low pressure monitor is configured to measure pressures slightly higher than those required by the blood pump 118. For example, a pressure of 50 mmHg or higher at a 100 mL/min flow rate may be maintained to avoid cavitation and/or vessel collapse. The pressure monitor could have an alarm setting for pressure drops of −50 to −100 mmHg to protect the blood pump 118 from cavitation and/or generate alerts for momentary kinks in the draw line 124 or other flow restrictions. An example of a pressure monitor includes an ICU Medical TransPac IV transducer coupled to a GE CareScape™ monitor.

FIG. 2B shows an example vasculature 402 of the patient 144 of FIG. 1. The draw line 124 is connected (e.g., via connector 138a) to a draw catheter 410 (e.g., the catheter may be introduced through a sheath, as described in relation to FIG. 2C). The catheter 410 accesses the vasculature through a radial artery 402 of the patient 144 and is advanced to the subclavian artery 404. The draw catheter 410 includes a hub 420 (which remains outside the vasculature). Hub 420a can be for a draw catheter 410, and hub 420b can be for a return catheter 412. The draw catheter 410 extends from the hub to a distal tip 422 of the draw catheter 410. A wire 408 and sensor module 406a-b may extend from the distal tip 422 and can operate in a manner described in relation to FIG. 2A. The draw catheter 410 is sized and configured to enable draw of a particular amount of blood (e.g., 100 mL/min) while enabling the blood pressure in the radial and/or subclavian arteries to be maintained above a threshold value to prevent vessel collapse that would substantially impede blood flow or drawing blood.

The draw catheter 410 is long enough to reach a subclavian artery 404 form the radial artery 402, such as from an insertion point 418 from the exterior of the patient near the elbow of the patient 144. The distal end of the draw catheter 410 is positioned in a subclavian artery 404 of the patient 144. The draw catheter 410 is sized to enable draw of a particular amount of blood (e.g., 100 mL/min) while enabling the blood pressure in the subclavian artery 404 to be maintained above a threshold value to prevent vessel collapse that would substantially impede blood flow or drawing blood. The draw catheter 410 may extend to the subclavian artery 404 at a length of up to approximately 60 centimeters. The draw catheter 410 extends to the subclavian artery 404 for drawing blood from the patient. In some implementations, the catheter 410 is less than 50 centimeters, and is used to reach from the radial artery 402 to the subclavian artery 404.

FIG. 2C shows an example vasculature 402 of a patient 144. A draw line 124 is connected (e.g., via connector 138a) to a draw catheter 424 (e.g., draw catheter 142 of FIG. 1). The draw catheter 424 extends from a catheter hub 420 (located outside of the vasculature) to a distal tip 422. The draw catheter 424 may be introduced via a sheath 416. A wire 408 and sensor module 406 may extend from the distal tip 422 and can operate in a manner described in relation to FIG. 2A. In this example, the draw catheter 424 extends to the radial artery, rather than all the way to the subclavian artery 404. In other implementations, the catheter may be inserted into other vessels upstream of the radial artery, e.g., the brachial, axillary, or subclavian artery.

The draw catheter 424 is inserted into the radial vasculature 402 through the sheath 416. The draw catheter 424 is advanced through the sheath 416 up to the catheter hub 420. In certain implementations, the sheath 416 may have a draw port connected to the draw line for drawing blood and the catheter 424 may serve as a return catheter for delivering gas-enriched blood to the vasculature, e.g., to coronary artery. This configuration allows for a single insertion point 418 to be used on the patient for both draw of the blood from the radial artery and/or other vessel downstream from the radial artery, e.g., subclavian artery, or a combination thereof and return of gas enriched blood to the vasculature, e.g., the coronary artery.

In some implementations, the catheters and/or the sheaths described herein may include an armored body. The armored body includes braided wire for flexibility and strength, with a medical grade plastic liner for containment of the catheter and/or sheath. Generally, the catheter and/or sheath is kink resistant. To be kink resistant, the catheter and/or sheath includes a wall thickness of about 8-12 thousands of an inch. In some implementations, the wall thickness is between 0.005 inches to 0.015 inches. In some implementations, the catheter and/or sheath includes an atraumatic tip such that there is a non-braided transition to the catheter or sheath tip. In some implementations, the draw catheter or sheath and the return catheter or sheath are combined into a single hybrid catheter or sheath including at least two lumens: a first lumen for drawing blood and a second, different lumen for retuning gas-enriched blood to the patient 144. For the hybrid catheter, an introducer sheath may or may not be used at the access point 418.

In the example of FIG. 2D, the draw catheter 412 and the return catheter 422 (shown in the subclavian, but may be advanced further upstream into a coronary artery), are each positioned in the same arm of the patient 144 such that both catheters access the vasculature via the radial artery 402 of the patient at insertion point 418. Optionally, the catheters may be inserted through a sheath 416 in the radial artery.

As described previously, the return catheter 422 is configured to return gas-enriched blood to the patient 144. The draw catheter 412 is configured to draw blood from artery 404 of the patient for gas enrichment in the blood circuit described in relation to FIG. 1. In this example, the access points for the catheters 424, 412 are the same access point 418 for accessing the radial artery 402 of the patient 144.

To enable the draw catheter 412 and return catheter 424 to be in the same arm of the patient 144, the return catheter 424 extends to the distal tip 422 in a subclavian artery 424 of the patient. The draw catheter 412 draws blood near the radial artery 402 of the patient. In certain implementations, the draw catheter may be advanced to and draw blood from vessels upstream of the radial artery, e.g., the brachial, axillary, or subclavian artery.

Exemplary draw catheters, e.g., as shown in FIGS. 2A-2C are configured for use with the delivery system 100 of FIG. 1. The draw catheters may be configured for operating with the delivery system 100 such as draw catheter 142. The draw catheters are configured to be inserted into the radial artery of the patient as previously described. In some implementations, the draw catheters described herein are configured for insertion or advancement into vessels upstream of the radial artery, e.g., the brachial, axillary, or subclavian artery, of the patient through the radial artery, as previously described.

Generally, an outer diameter and length of the catheters are sufficient to support a blood flow rate or predetermined blood flow rate of 50-150 mL/min while avoiding a pressure drop that would cause pump cavitation. Example geometries of such catheters are shown in Table 1 below.

TABLE 1
Draw Pressure at Blood Pump based on guide catheter
size and length (100 ml/min flow rate) (assuming
arterial pressure 80 mmHg nominal)

| Catheter | 6 Fr Guide Draw | 7 Fr Guide Draw |
|---|---|---|
| Length (cm) | Pressure (mmHg) | Pressure (mmHg) |
| 0 | 30 | 30 |
| 10 | 12 | 19 |
| 20 | −7 | 8 |
| 30 | −25 | −3 |
| 40 | −44 | −14 |
| 50 | −62 | −25 |
| 60 | −81 | −35 |
| 70 | −99 | −46 |
| 80 | — | −57 |
| 90 | — | −68 |
| 100 | — | −79 |

In some implementations, the catheter has an outer diameter that is 6-7 French. The catheter length may be 10-100 cm. Depending on the length of the catheter, the pressure drop caused by blood draw at a rate of 100 ml/min, from the distal end of the catheter to the blood pump, may be between 0 mmHG to negative 180 (−180) mmHG. In certain implementations, a pressure monitor may provide an alert of a presser drop from −50 to −100 mmHG.

The draw catheters described herein can each be configured as follows. In some implementations, the one or more lumens of the catheter can comprise a braided pattern. In some implementations, the braided pattern comprises a rectangular cross section. Generally, the braid is flexible but is still firm to prevent kinking. Generally, the one or more lumens of the catheter each comprise a wall thickness between 0.005 inches to 0.015 inches. The wall thickness prevents kinking of the one or more lumens of the catheter. In some implementations, the one or more lumens of the catheter comprise an atraumatic tip. An atraumatic tip includes a shape and material to cause minimal tissue injury. For example, the atraumatic tip can be rounded. The atraumatic tip can taper to a rounded edge. The atraumatic tip can include a soft, pliable material that does not pierce the radial artery 402. In some implementations, the catheter includes a distal band on a distal portion of the catheter. The distal band aligns with a predetermined location in the radial artery or a vessel upstream of the radial artery, e.g., the brachial, axillary, or subclavian artery of the patient to assist a user in determining how far to advance the catheter into the artery of the patient during use for proper positioning, e.g., either in the radial artery or near the subclavian artery.

In some implementations, the catheter is supported by a sheath configured for inserting into the radial artery of the patient. The sheath can include a braided wire and a plastic liner over the braided wire. Generally, the braid is flexible but is still firm to prevent kinking. The braid can include a wire that is encapsulated (e.g., extruded or overmolded) with medical grade plastic (e.g. Pebax™). Generally, for the sheath, a flat braid provides a rectangular, thinner cross section relative to a round braid. A rounder braid causes a relatively thicker wall for the sheath. Generally the sheath is between ˜50-100 centimeters in length.

FIG. 2E shows an example vasculature 400 of the patient 144 of FIG. 1. The draw line 124 is connected (e.g., via connector 138a) to a draw catheter 410 (e.g., draw catheter 142 of FIG. 1). The draw catheter 410 is inserted in a radial artery 402a of the patient 144 and advanced to a subclavian artery 404 to draw blood at a subclavian artery 404. The draw catheter 410 is sized to enable draw of a particular amount of blood (e.g., 100 mL/min) while enabling the blood pressure or change in blood pressure in the radial artery 402a to be maintained above or below a threshold value. The blood circuit therefore includes drawing blood at the subclavian artery 404, as described in relation to FIG. 2D, and returning blood to the patient in a radial artery 402b as described in FIG. 2A. This combination of radial return and subclavian draw of blood can help reduce the likelihood of collapsing the radial or subclavian artery and substantially impeding blood flow.

FIGS. 3A-3B show an example catheter 500 configured to prop or hold open a radial vessel 402 of the patient. For example, in FIG. 3A, the catheter 500 enters in a straight configuration. A shape memory of the catheter 500 allows the catheter body 502 to coil once in the vasculature 402 to keep the vessel propped open during blood draw from the vessel. The catheter body 500 is advanced along axis 504 in a straight configuration. In FIG. 3B, the catheter body 502 is shown in a coiled configuration allowing it to maintain the vessel 402 open during blood draw, maintaining the vessel at diameter D1. The coiled catheter body 502 can prevent the vessel 402 from collapsing to a degree that would substantially impede blood flow when blood is drawn by the catheter 500. In certain implementations, where the catheter is advanced to a vessel upstream of the radial artery, e.g., the brachial, axillary, or subclavian artery, the respective artery may be held or propped open by the coiled catheter to avoid collapsing to a degree that would substantially impede blood flow or drawing blood.

In some implementations, e.g., where the catheter is configured to draw blood at the radial artery without advancement to the subclavian artery, the radial artery may be propped open such as by a sheath or by the catheter body. For example, the catheter body may have a spiral or coiled shape to prop open the radial vessel, in which blood flows through a center or central portion or lumen of the catheter body. In some implementations, the catheter comprises a straight lumen. In some implementations, the catheter comprises a coiled lumen configured to prop open a vessel of the patient. In some implementations, the catheter comprises a straight configuration upon insertion and assumes a coiled configuration inside the artery or vessel. In some implementations, the catheter body is formed from a shape memory material (e.g., nickel titanium, such as Nitinol). In this example, the catheter body may be inserted into the radial artery in a straight configuration. Once inserted, the catheter is configured to coil and prop the radial artery vessel open to prevent collapse during blood draw (e.g., as shown in FIGS. 3A-3B). In certain implementations, where the catheter is advanced to a vessel upstream of the radial artery, e.g., the brachial, axillary, or subclavian artery, the respective artery may be held or propped open to avoid collapsing to a degree that would substantially impede blood flow or drawing blood.

Returning to FIG. 1, the draw line 124 may include a bubble trap chamber 120 and is configured to interface with a pump 118 and may be configured to interface with a first flow control mechanism, e.g., a draw line flow control mechanism 122 of the delivery system 100. Pressure transducers 138a-b may be located on either side of the pump 118 to measure pressure of blood flowing through the blood circuit, such as through the draw line 124, through the return line 130, or through each of the draw line and the return line.

The delivery system may include a flow sensor 146, for example on or near the blood circuit (e.g., on the draw line (not shown) or return line 130) to measure the flow rate of the blood circulation in the blood circuit. For example, the flow sensor 146 can measure a number of milliliters per minute (mL/min) of blood drawn or gas-enriched blood delivered to the patient 144. In some implementations, the flow sensor 146 is positioned near the pump 118. In some implementations, the flow sensor is positioned near the return line 130 or near connectors 138a or 138b.

To deliver gas-enriched blood to a patient 144, the delivery system 100 operates as follows. The delivery system 100 console 102 is connected to each other component of the delivery system. For example, the cartridge 200 is inserted into the cartridge housing 104 of the console 102. Tubing (e.g., the draw and return lines 124, 130) extending from the cartridge and connecting the cartridge 200 to the catheter 136 may be interfaced with the draw flow control mechanism 122, and/or return flow control mechanism 128, and pump 118 of the console. The cartridge and draw and return lines 124, 130 may be configured such that upon insertion of the cartridge into the cartridge housing, the tubing automatically self-aligns with the draw flow control mechanism 122 and/or return flow control mechanism 128, and/or pump. For example, the cartridge may have return and draw lines, which have a predefined orientation and shape that match with a corresponding shape or design in the cartridge housing and/or on the console. The predefined orientation and shape is such that upon insertion of the cartridge into the cartridge housing, the draw line and return line automatically align with and interface with the draw and/or return flow control mechanisms 122, 128, and the pump 118. The power supply 114 is connected to an external power source for providing power to the console 102. The oxygen supply 110 receptacle is provided an oxygen bottle 112 for providing the source of oxygen to the cartridge 200. The user interface 132 can indicate whether any of these consumables are missing from the delivery system 100 before or when delivery of the gas-enriched blood to the patient is beginning. In certain implementations, the cartridge and draw and return lines may be aligned by the user

Once each of the components of the delivery system are connected, including one or more of the cartridge 200, pump 118, bubble trap 120, bubble detector 126, draw flow control mechanism 122, return flow control mechanism 128, and catheter 136, the delivery system 100 is ready for use. The blood circuit is shown with arrows representing the direction of blood flow during operation of the delivery system 100, where blood is pulled from the catheter 136 through the draw line, through the cartridge and returned to the catheter via the return line.

Prior to operation, a priming process is run which causes the blood circuit to be filled or substantially filled to a threshold level with blood such that there is no room air and/or air bubbles in the blood circuit, which could travel to the patient 144. For example, the draw line 124 and return line 130 are filled with blood. For example, the bubble trap 120 and pump 118, and the tubing connecting the various elements of the blood circuit, are filled with blood. The blood mixing chamber of the cartridge 200 is filled with blood, e.g., to a threshold level.

Room air and/or air bubbles from each of the elements of the blood circuit is vented from the respective elements, as subsequently described. The bubble detector 126 is configured to detect any bubbles present in the blood circuit during operation of the delivery system 100 and can send a signal resulting in the closing of the return flow control mechanism 128 if room air and/or air bubbles are detected in the blood circuit. This prevents air bubbles from reaching the patient 144 at the catheter 136. The bubble detector 126 can include an ultrasonic sensor, infrared (IR) sensor (e.g., a photogate), or other such mechanism for detecting air or bubbles in line. For example, the bubble detector 126 can include an IR sensor that senses an IR beam sent through the fluid of the blood circuit. An air bubble in the fluid distorts the beam, which can be detected by an IR sensor.

The delivery system 100 may be configured to control the oxygen levels in the blood and/or tissues of the patient 144 by controlling the oxygen levels in the supersaturated oxygen liquid or solution, (e.g., targeting a dissolved O₂ concentration in saline of 0.1 ml O2/ml liquid (STP) or greater or 0.1-6 ml O₂/ml liquid (STP) or 0.2-3 ml O₂/ml liquid (STP) and/or the flow rate of the supersaturated oxygen enriched blood delivered to the patient 144, e.g., by controlling the speed of the pump to achieve a target blood flow rate of ml/min, 30-300 ml/min or 50-150 ml/min. The system 100 may be configured to titrate oxygen into liquid e.g., saline, to be mixed with blood and adjust the oxygen level and/or blood flow rate, until the desired oxygen level is achieved (e.g., as measured by a blood oxygen sensor in the patient 144). In an example, the concentration of oxygen delivered, and/or blood flow rate may be modulated during treatment based on feedback from one or more sensors measuring various patient and/or system parameters.

One example of a sensor for measuring a partial pressure (pO₂) of oxygen or oxygen saturation SO₂ in the patient's blood is a pulse oximeter. A pulse oximeter may be used for estimating arterial pO₂ or SO₂. Pulse oximetry estimates the percentage of oxygen bound to hemoglobin in the blood. A pulse oximeter uses light-emitting diodes and a light-sensitive sensor to measure the absorption of red and infrared light. In another example, a sensor for measuring partial pressure of oxygen comprises an electrode such as a Clark electrode for measuring pO₂. A Clark electrode is an electrode that measures ambient oxygen concentration in a liquid using a catalytic platinum surface according to the net reaction O₂+4 e⁻+4 H+→2 H₂O. The various sensors may be coupled to a controller of the system via a cable or other wired connection or via a wireless connection.

As discussed herein, one or more blood pressure sensors may be used to measure blood pressure values in blood from a patient receiving gas-enrichment therapy. A processor of the controller receives signals from the blood pressure sensor, which correspond to the measured values of blood pressure and changes in blood pressure. The processor may compare the measured blood pressure to a target range of blood pressure, e.g., blood pressure in a healthy individual. The processor may generate an alert, e.g., through the user interface, that indicates the blood pressure or a change in blood pressure. The measured blood pressure or change in blood pressure may indicate the effectiveness of the supersaturated oxygen or gas therapy, letting the caregiver know if the blood pressure is within a target range in order to optimize the SSO2 therapy. The processor may control the gas enrichment system by modifying one or more saline or blood parameters in the gas-enrichment system to optimize therapy based on the blood pressure feedback.

A change in blood pressure may be indicative of a change in blood flow in myocardial tissue in response to the gas-enrichment therapy. The gas-enrichment therapy, e.g., SSO2 therapy, provides a high concentration gradient of O2 that enables increased diffusive transfer to ischemic areas of myocardium. This diffusive transfer of O2 to areas most in need does not depend on blood flow and thus O2 can easily access the endothelial cells of capillaries suffering from edema (swelling). SSO2 therapy is able to reverse this edema response in the microvasculature and restore flow, nurturing surrounding heart tissue with oxygenated blood.

In addition to the wire with pressure sensors discussed above, another example sensor for measuring an arterial pressure of the patient's blood includes a pressure sensor positioned in or coupled to the catheter. The catheter may be connected to a fluid-filled system or pressure tube, which is connected to an electronic pressure transducer and/or pressure monitor. A change in detected blood pressure may be indicative of improved perfusion and/or restored flow in ischemic tissue as a result of the SSO2 therapy. The therapy may result in improved heart function. In certain implementations, the processor may control the delivery of SSO2 therapy based on the arterial pressure feedback.

In certain implementations, feedback may be based on a measured blood pressure waveform. A change in a waveform reflection pattern may be detected. In one example, changes in the reflection pattern of the normal pulsatile waveform of the patient's blood pressure may be detected or measured. In another example, a pulsatile flow may be created (for more fine tuning), and changes in the reflection patter of the created pulsatile waveform of the patient's blood pressure may be detected or measured. In either example, the pulsatile waveform may be analyzed for information, such as the relative magnitude and the timing of the secondary peak identified in that waveform.

The processor of a controller 102 can receive the signals from these sensors, which signals correspond to the measured values of pO₂. The processor compares the measured pO₂ to a target range of blood pO₂, e.g., 760-1500 mmHg. The target range can be calculated based on a source input blood pO₂ of 80 mmHg, a blood flow rate of 50-150 ml/min, an SSO₂ saline flow rate of 2-5 ml/min and dissolved O₂ concentration in saline of 0.2-3 ml O₂/ml saline (STP). The controller can adjust any of the above parameters based on the measured pO₂ in blood to achieve an arterial blood pO₂ within the target range. The processor may generate an alert, e.g., through a user interface, audible alarm and/or visual alarm that indicates the level of pO₂. The measured pO₂ indicates the effectiveness of the supersaturated oxygen therapy, letting the caregiver know if the pO₂ in blood is within the target range for optimizing the delivery of oxygen to the patient's ischemic tissue. In certain implementations, the processor may control the delivery of supersaturated oxygen therapy by modifying one or more of the above referenced saline or oxygen parameters based on the signals received from the sensors.

Another example of a sensor is an O₂ fluorescence probe. The fluorescence probe may be coupled to a controller of the system via a cable or other wired or wireless connection. A light source of the O₂ fluorescence probe is illuminated. A fiber optic cable can be used to provide light to the light source in certain implementations, where the fiber optic cable is connected to the controller of the system. The fluorescence of a sensor molecule of the O₂ fluorescence probe is measured. The sensor molecule can include fluorophore. A signal is received by the processor from the O₂ fluorescence probe based on the fluorescence measurement. Fluorescence is measured by measuring the lifetime or decay of the fluorescence intensity signal from the illuminated sensor molecule (e.g., fluorophore) on the fluorescence probe. The decay of this signal is caused by the quenching effect of oxygen molecules in the blood or in tissue on the fluorescence intensity signal of the sensor molecule. The processor can determine the oxygen concentration, SO₂ or pO₂ in blood or tissue based on the quenching effect of oxygen on the florescence intensity signal of the florescence probe. Changes in a time that is required for the signal to decay due to oxygen quenching are indicative of the local oxygen concentration, SO₂ or pO₂ in blood or tissue. The processor generates an alert, e.g., through a user interface, audible alarm and/or visual alarm, based on the determined oxygen concentration, SO₂ or pO₂ in blood or tissue. The alert may indicate the effectiveness of the supersaturated oxygen therapy. The determined oxygen concentration, SO₂ or pO₂ indicates the effectiveness of the supersaturated oxygen therapy, letting the caregiver know if the oxygen concentration, SO₂ or pO₂ in blood is within a predefined target range (e.g., the expected range for a healthy individual) for optimizing the delivery of oxygen to the patient 144. In certain implementations, the processor may control the delivery of supersaturated oxygen therapy by modifying one or more of the saline or oxygen parameters, e.g., saline flow rate or dissolved O₂ concentration in saline, based on the determined oxygen concentration, SO₂ or pO₂ values.

The user interface 132 is configured to display operational data and/or patient data on the user interface in a configuration that allows a user to determine a status for the SSO₂ liquid and gas-enriched blood delivery to the patient 144. The user interface 106 shows a current operational status of the delivery system 100.

These values can be stored as a time sequence of data entries or log entries in an operational log. The user interface may include a visual representation of the operational log, the visual representation including operational data specifying how the delivery system 100 is performing during delivery of the enriched blood to the patient. For example, the delivery system 100 logs sensor readings during delivery and generates an alert or report indicating whether the delivery of the gas-enriched blood should be titrated. In some implementations, the delivery system 100 can send logged data to remote, networked storage (e.g., in cloud storage) for access from one or more networked devices.

In some implementations, various data elements are logged during the delivery process. For example, the duration of delivery can be logged. Each time a checkpoint is reached, a time stamp associated with the checkpoint is saved. Checkpoints can include completion of the delivery process, indication of titration of the delivery of gas-enriched blood, indication of values of one or more physiological parameters such as pressure or flow rate, a visualization of an estimate of the microvascular resistance, or any other data of interest during the delivery process. The values of sensors, such as the level sensors, pressure sensors and temperature sensors, can be stored at given instances in time. The operational values of devices on the blood circuit can be monitored, such as how fast the pump is operating, blood levels in the blood mixing chamber or bubble trap, when one or more flow control mechanisms 122, 128 are actuated, and so forth. These data provides information to determine whether an issue is occurring during delivery of the gas-enriched blood.

In some implementations, the delivery system 100 may include a processor, a memory, and associated circuitry coupled to the one or more sensors for detecting physiological data. The physiological data is collected and/or stored in the system for retrospective, current or other review. The delivery system 100 is configured to generate log entries for the operational data (e.g., delivery data). The log entries may be displayed on the user interface 132. In certain implementations, the log entries can each be structured messages that include particular values associated with the operation of the delivery system 100, generated from data messages. In some implementations, a data message (also called a log message) represents an instant snapshot of the operational data. For example, a data message can include treatment data or current pO₂ and SO₂ values at a given time (e.g., associated with a time stamp). In some implementations, a data message can include data representing a treatment period or system mode of the gas-enriched liquid treatment for the patient 144 in a structured log entry. The data messages are stored in a digital format that enables streaming of the data messages to a remote system. The remote system is configured to quickly extract the values representing the patient data and the operational data of the delivery system 100 and display a representation of these data on a local or remote user interface. For example, data messages can be formatted for streaming to an operator or nurse's station from a hospital room. In some implementations, data messages can include warnings or alerts that prompt intervention from a user of the remote system. In some implementations, the data messages can be stored in a structured format that facilitates searching and retrieving of operational data for the patient 144 for operation of the delivery system 100 during SSO₂ delivery.

In some implementations, the log entries can each be structured messages that include particular values associated with the operation of the delivery system 100, generated from data messages. For example, the data messages can indicate a current snapshot of the operation of the delivery system 100. In this case, the values of the data message include a list of operational values and/or physiological values. The operational values and/or physiological values can be parsed from the data messages (e.g., by a remote device) and used to populate a screen or display of a remote computing system. For example, the delivery system 100 can transmit a stream of data including the data messages to a remote system for remote monitoring of the operation of the delivery system 100. In some implementations, the processor is configured to stream digital output data having the patient data and the operational data to a remote server. In some implementations, operational and patient data may be transmitted or streamed in real time or near real time via a wired, RS-232 streaming output on the system console to a remote processor or computer, e.g., to an EMR data hub or hospital hub. In some implementations, operational and patient data may be transmitted or streamed in real time or near real time over a WiFi communications, Bluetooth, cellular, USB or other wireless connection or link.

The data messages can include summary data. For example, log entries can include data representing a summary of operational and/or physiological data for a time period (e.g., pre-titration data, titration data, and post titration data). Each log entry may form all or a portion of the operational log, which provides an overall summary of the operation of the delivery system 100. The operational log allows a medical service provider to quickly review the summary of the operation of the delivery system 100. The operational and/or physiological data, e.g., data messages, log entries, operational log and/or other data, stored by the system processor or an accessary to the system or data module, coupled to the system console, may be stored on volatile or non-volatile memory. The log entries can be visually represented on the user interface 132.

Data messages may provide instant values of operational data of the delivery system 100 and the physiological data. Log entries may represent data gathered over time and can be part of a system and/or patient profile. For example, the operational log and the log entries can be stored in electronic medical records (EMR).

In some implementations, the log entries of the operational log are transmitted to a remote device (such as a data hub in a hospital). The delivery system 100 sends the data including the log entries to the remote device in one or more different ways. The delivery system 100 sends the log entries data to a remote device in response to a trigger. For example, the delivery system 100 can send the log entries to the remote device once titration of delivery of the gas-enriched blood is completed. In some implementations, the delivery system 100 sends the operational log data once all treatment is completed. For example, when the cartridge 200 is removed or the pump 118 is powered off, the controller 102 can determine that treatment is completed and send the log entry data to the remote device.

In some implementations, the delivery system 100 sends the operational log data to the remote device upon detecting a fault, such as a bubble trap 120 fault, a catheter 136 fault, a patient SO₂ or pO₂, or blood pressure or flow rate value failing a threshold, etc. The operational log data can be analyzed (e.g., by a user) to determine why the fault occurred and/or to determine whether operation of the delivery system 100 is adversely impacted by the fault. This enables the user to take corrective measures immediately (e.g., replacing a bubble trap 120, fixing a fluid leak, etc.) to ensure that treatment of the patient 144 is not compromised.

In some implementations, the delivery system 100 sends the operational log data without a trigger. For example, the delivery system 100 can send the log entry data to the remote device periodically (e.g., once per minute, once per hour, and so forth).

In an aspect, the delivery system 100 links the log entries related to operation together in a structured format. For example, a key value can be stored with each log entry. The entire log of the operation of the delivery system 100 can be retrieved by referencing the key value.

The delivery system 100 can generate one or more alerts to indicate a status of one or more components of the delivery system 100. The alerts can be generated based on the operational log data or data of the data messages. The alert can be generated for presentation on a user interface 132 of the delivery system 100. The processor may send the alert to one or more other computing devices, such as computing devices associated with a health care provider of the patient 144. In an aspect, a user interface is configured to communicate with the processor, wherein the data representing the alert indicating whether a fault has occurred, priming has initiated/completed, or any other relevant aspect of the operation of the delivery system 100 that satisfies a notification rule causes a notification to be displayed on a user interface. The user interface may be coupled to the console via a wire or wirelessly (e.g., the user interface may be a portable tablet or remote computing device)

The alert may indicate that there is a fault or error in operation of the delivery system 100. The alert provides an indicator for a health care provider to investigate the operation of the delivery system 100, such as to investigate whether any faults have occurred. The alerts may indicate that titration of the delivery of the gas-enriched blood has completed, that there is a pressure over value in the blood circuit, that room air and/or air bubbles have been detected, etc.

In some implementations, the processor generates the alert to cause one or more devices to perform an action. For example, feedback can be presented to a healthcare provider, such as an audio cue, visual presentation, and so forth. The alert can cause a device to contact a healthcare provider (e.g., place a phone call or page to a physician, nurse, etc.). The alert can cause a device to display particular data about the delivery process or performance of the system, or data about the patient 144, such as a presentation of the patient's SO₂ and/or pO₂ or blood pressure or flow rate values over a given treatment period. The alert can cause a device to update a health record associated with the patient 144 or cause the device to retrieve a health record associated with the patient for further analysis. In certain implementations, the processor of the system may be configured to determine if the alert is a real time alert or recorded for retrospective review. If it is a real time, the processor determines whether to display the alert on the user interface, transmit the alert in an information chain, or send the alert data to a third-party monitor. An example route is to send the alert to a physician or nurse's cell phone.

The alert may open a cell phone-based application or open an Internet-based application. From either application the physician or nurse could see the alert plus other relevant data that may have been transmitted. The alert may include a hospital specific patient identifier, but otherwise be invisible as to the identity of the patient 144, unless the physician or the hospital has added the patient's name to either the application on their phone or to the Internet. The alert may include a non-patient specific identifier such as a bed number. Additionally, the physician would have the opportunity to take actions in response to receiving the alert. This might include triggering a phone call to the ICU desk or marking that the physician has seen the alert. Changing the duration or range of a monitored value would allow the user to set a duration so that a transient spike would not trigger the alert. In the case of adjusting the time and/or duration of the alert, such an adjustment may only affect the notification to that specific person.

A dual alert to a nurse or physician might have different alert ranges and actions. The described features may put the user, e.g., physician in complete control. For example, the first point of control may be at the bedside, where the alert ranges may be set. The second point of control may be at the receiving application or website where the user may adjust nominal settings, e.g., for “tones”. As such, two or more triggers may be established: the first is to “send” the alert from the machine into the network to the receiving device; and the second is the action that the receiving device takes upon receiving the alert. A scheduling feature may also be provided that allows for the transfer data from one physician going off shift to another coming on shift. A response tree may be provided that requires an acknowledgement that the alert has been seen or transferred from one physician to another. For example, a first doctor is given 5 minutes to acknowledge receipt of the alert, and if no acknowledgment is made, the alert is sent to another physician or nurse. In certain implementations, one or more of the various alerts or alert parameters described herein may be customized by the user. Multiple options for alert delivery, e.g., device display, nurse's station, EMR, cell phone, etc. may be set. An alert for thermoregulatory activity of a patient 144 may include other forms. For example, a color scale or audible alert may be output via the user interface to provide a value indicative of patient activity.

In some implementations, a medical service provider can query the delivery system 100 to obtain the operational data. The query can request particular data, such as what the battery status is, determine whether titration of gas-enriched blood was successful, and so forth.

In some implementations, a controller is configured to store digital output data representing the delivery process in a data store. The controller is configured to detect that a trigger condition of the delivery process is satisfied. For example, the trigger condition can include completion of all or a portion of the delivery process. In some implementations, the controller, in response to detecting that the trigger condition is satisfied, transmits the digital output data to a remote device in real time or in near real time, e.g., during or after the delivery of gas-enriched blood by the delivery system.

In some implementations, the digital output data includes a predefined format that enables the digital output data to be streamed to a remote device. The delivery system can include a transmitter configured to transmit the digital output data to the remote device. In some implementations, the predefined format is configured to enable the remote device to parse the digital output data for displaying the operational SO₂ or pO₂ or blood pressure or flow rate data and/or the operational data upon receiving the digital output data. In some implementations, the process includes streaming the digital output data over a WiFi communications, Bluetooth, cellular, or other wireless connection or link or USB. In some implementations, the process includes transmitting the digital output data over a wired connection.

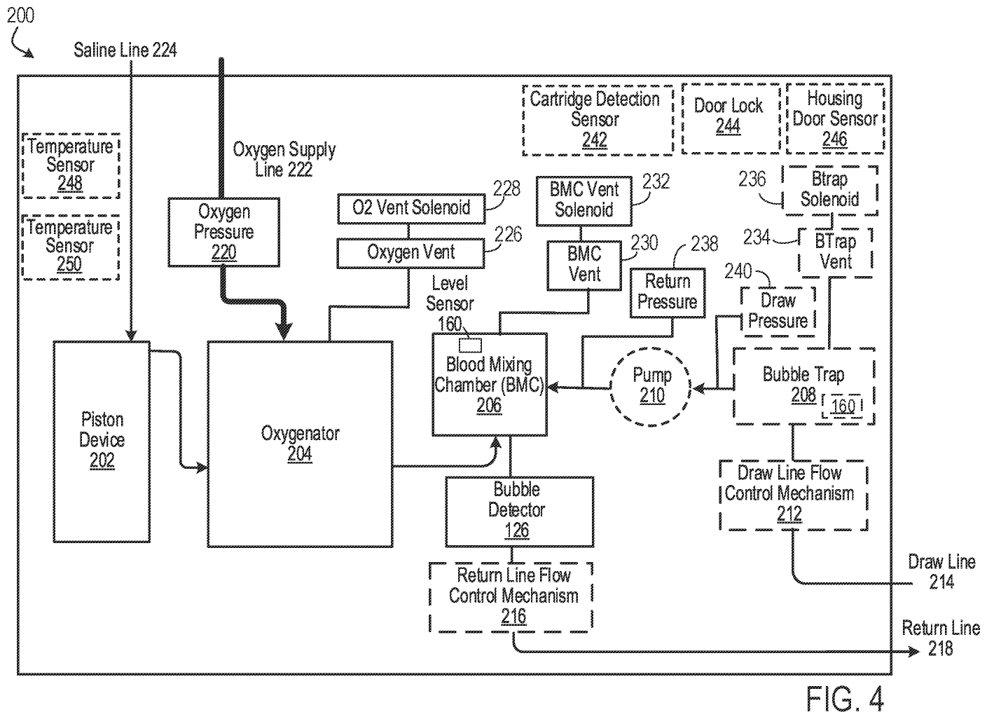

FIG. 4 is a diagram of an example of a portion of the system of FIG. 1 including the cartridge 200. In this example, the cartridge 200 includes a fluid supply chamber (piston device 202), a gas enrichment chamber (an oxygenator 204), and a blood mixing chamber 206. In some implementations, the cartridge 200 may also include a bubble trap 208, and at least a portion of the draw line 214 tubing and the return line 218 tubing. In FIG. 4, the pump 210 is similar to pump 118, the draw line 214 is similar to draw line 124, the return line 218 is similar to return line 130, and the bubble trap 208 is similar to bubble trap 120. The cartridge 200 is consumable portion of the blood circuit that includes portions of the blood circuit that contact the patient's blood. The return draw flow control mechanism 216, pump 210, and draw flow control mechanism 212 are shown in dashed lines because these are a part of the console system and are reusable. Similarly, the return pressure sensor 238 and/or the draw pressure sensor 240 are reusable; however, in certain embodiments, the return pressure sensor 238 and/or the draw pressure sensor 240 may be part of the single use consumable cartridge and tubing. In some implementations, the return pressure sensor 238 the draw pressure sensor 240, the bubble trap 208, and/or the draw line flow control mechanism 212 are optionally included in the delivery system 100 or in the blood flow circuit.

The cartridge 200 is configured to interface with components of the console 102 of the delivery system 100 during operation, priming and treatment. A portion of the tubing of the cartridge 200, which can be called a pump tube, is configured to be placed in the pump 210 of the console. The draw line 214 tubing and the return line 218 tubing are oriented to be placed inside the draw flow control mechanism 212 and the return flow control mechanism 216, respectively. The flow control mechanisms 212, 216 are coupled to the console 102. When the cartridge 200 is installed, the flow control mechanisms 212, 216 align with the draw and return lines 214, 218 to enable the flow control mechanisms to restrict fluid flow (e.g., by clamping) in the draw and return lines 214, 218. The draw flow control mechanism 212 and the return flow control mechanism 216 are actuated by control signals of a controller of the console 102. Similarly, the pump 210 is coupled to the console 102. The pump 210 is activated by control signals of the controller of the console for pumping in either the draw line direction or the return line direction as needed.

The piston device 202 includes a mechanical device for drawing saline from the fluid source. The fluid from the IV source is drawn through tubing into a piston chamber. The piston moves vertically in the chamber based on signals from a piston actuator. A load cell determines the force required to move the piston. A stepper motor controls the motion of the actuator. An encoder reports the piston position based on the stepper motor rotor location. A piston top sensor and piston bottom sensor can detect when the piston moves to an edge of the chamber. The position of the piston determines how much fluid from the saline bag is sent to the oxygenator.

The piston device 202 is configured to draw saline into the oxygenator 204. The oxygenator 204 is configured to add oxygen to the saline from the saline bag 106. An oxygen pressure line 220 adds oxygen to the oxygenator 204. The oxygenator 204 is coupled to an oxygen vent 226 and an oxygen vent solenoid 228 that controls operation of the vent 226. The oxygenator vent 226 is configured to vent excess air from the oxygenator if the oxygen pressure exceeds a threshold value.

The oxygenator 204 includes an oxygen chamber, an atomizer, and a valve manifold. The valve manifold includes several valves such as a fill valve, a flush valve, and a supersaturated oxygen SSO₂ flow valve (not shown). Each of the fill valve, flush valve, and SSO₂ flow valve are controlled by a respective solenoid. A fill solenoid opens/closes the fill valve. A flush solenoid opens/closes the flush valve. A SSO₂ flow solenoid opens/closes the flow valve. An SSO₂ level sensor 400 indicates a level of the gas-enriched liquid in the oxygenator.

The oxygen chamber is connected to the oxygen pressure line and the oxygen vent. The oxygenator releases excess oxygen through oxygen vent 426 and receives additional oxygen through oxygen pressure line. The oxygenator receives fluid from the piston chamber. The atomizer includes a central passageway in which a one-way valve is disposed. When the fluid pressure overcomes the force of the spring in the one-way valve and overcomes the pressure of the oxygen within the atomizer chamber, the fluid travels through the passageway and is expelled from a nozzle at the end of the atomizer.

The nozzle forms fluid droplets into which the oxygen within the atomization chamber diffuses as the droplets travel within the atomization chamber. This oxygen-enriched fluid is referred to a SSO₂ solution. The nozzle is preferably a simplex-type, swirled pressurized atomizer nozzle including a fluid orifice of about 0.004 inches diameter to 0.005 inches diameter. The droplets infused with the oxygen fall into a pool at the bottom of the atomizer chamber. Since the atomizer will not atomize properly if the level of the pool rises above the level of the nozzle, the level of the pool is controlled to ensure that the atomizer continues to function properly. Once the oxygen has been dissolved into the saline using the controlled pressure, the gas-enriched saline is sent to the blood mixing chamber 206 for mixing with blood in the blood circuit.

The blood mixing chamber 206 is connected to the oxygenator 204. The blood mixing chamber 206 is thus a part of the blood circuit. The blood mixing chamber 206 is positioned between the pump 210 tubing and the return line flow control mechanism 216 and bubble detector 126. A blood mixing chamber vent 230 is configured to vent any room air and/or air bubbles from the blood mixing chamber 206. A blood mixing chamber vent solenoid 232 controls operation of the vent 230.

The blood mixing chamber 206 includes a volume configured to receive gas-enriched saline from the oxygenator 204. The blood mixing chamber includes low sensor and a high sensor. The low sensor is configured to detect when the blood mixing volume 502 is empty. The high sensor detects when the blood mixing volume is full.

The blood mixing volume vents room air and/or air bubbles from the blood circuit through the vent through the line. The blood mixing chamber receives gas-enriched saline from the oxygenator. The blood mixing chamber receives blood from the pump 210 from the pump tube during operation of the delivery system 100. The gas-enriched saline from the oxygenator 204 mixes with the blood from the draw line of the blood circuit. A return pressure sensor measures pressure in the blood circuit on the return line side of the pump 210. The blood from the blood circuit passes through the blood mixing volume and mixes with the gas-enriched saline from the oxygenator 204. The return line draws blood out of the blood mixing volume to the bubble detector 126.

The blood mixing chamber 206 oxygenator and piston chamber may be located in a single housing or separate from one another. The pump 210 is configured to interface with a pump tube. The pump tube connects the bubble trap 208 to the pump 210. The pump tube connects the blood mixing chamber 210 to the pump on of the opposite side of the pump 210 from the bubble trap 208. Blood in the blood circuit during operation of the delivery system 100 thus comes from the draw line 214 through the bubble trap, is pumped by the pump 210, goes through the blood mixing chamber 206, and then goes through or passes by the bubble detector 126 in the return line 218.

A bubble trap 208 may be provided and configured to remove room air and/or air bubbles from the blood circuit. The bubble trap 208 has a bubble trap volume configured to receive blood from the draw line. The bubble trap volume vents room air and/or air bubbles from the volume to the bubble trap vent. Bubbles rise to the top of the volume and are vented. The bubble trap volume has a low sensor to detect when the bubble trap volume is empty. The bubble trap volume has a high sensor to detect when the bubble trap volume is full. When the volume is full of blood, the bubble trap 208 is primed. Venting of the bubble trap 208 through a bubble trap vent 234 can be controlled by a bubble trap solenoid 236, which is actuated for venting the bubble trap.

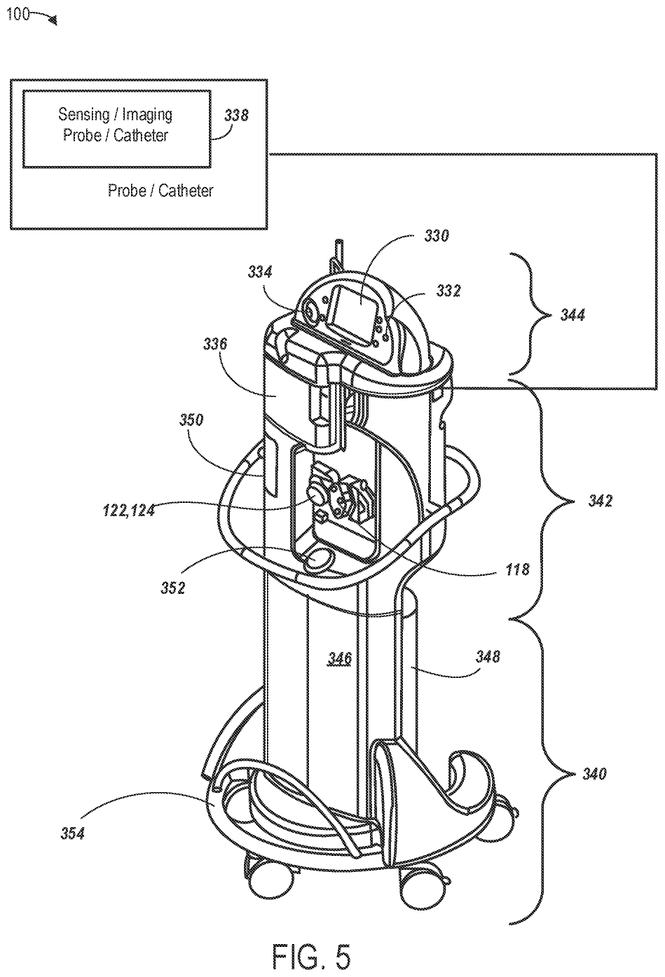

FIG. 5 shows the system 100 of FIG. 1 for administering SSO₂ gas-enrichment therapy, e.g., SSO₂ therapy in greater detail. The system 100 for administering SSO₂ therapy generally includes three component devices: the main control system, the gas enrichment system (e.g., oxygenation cartridge), and the infusion device (e.g., an infusion catheter). These devices function together to create a highly oxygen-enriched saline solution called SSO₂ solution. Blood is mixed with the SSO₂ solution producing supersaturated oxygen enriched blood. The supersaturated oxygen enriched blood is delivered to the patient. The system 100 may have a modular design comprising three removable modules such as a base module 340, the mid-section control module 342, and the display module 346. The system 100 may also have a sensing catheter 338, which can be implemented via a catheter (e.g., catheter 136) in accordance with certain implementations, or may have other sensing or imaging inputs. A gas tank receptacle 346 is provided on the backside of the base module 340 for receiving and housing a standard “B-bottle” USP oxygen tank 348. The oxygen tank 348 is mounted to the system via a gas tank adapter. A suitable gas, such as oxygen, is delivered from the oxygen tank 348, to a second chamber within an oxygenation cartridge. The physiologic liquid, e.g., saline, from a first chamber is pumped into the second chamber and atomized to create a supersaturated oxygen enriched physiologic solution. This supersaturated oxygen enriched physiologic solution is then delivered into a third chamber of the oxygenation cartridge along with the blood from the patient. As the patient's blood mixes with the supersaturated oxygen enriched physiologic solution, supersaturated oxygen enriched blood is created and then delivered to a targeted major epicardial artery, e.g., the left main coronary artery, via an infusion catheter.

Each of the three modules 340, 342, 344 of the system 100 may include doors or access panels for protecting and accessing the various components housed therein. For example, the mid-section control module 342 includes a hinged door 336 for enclosing the gas-enrichment system (i.e. the cartridge) and access panel 350 for covering the access window to the internal space of the module. A safety switch (e.g. an emergency stop switch 352) may be provided so that a user can initiate a shutdown of the system in the same fashion even if the system is operating within its prescribed bounds.

In the above particular embodiment, the body of the base module 340 is made up of a tubular chassis situated on a circular-shaped pedestal 354. A plurality of wheels are mounted on the bottom of the circular-shaped pedestal to provide mobility for the system. The wheels have a locking mechanism for keeping the wheels stationary. The base chassis houses certain electrical and mechanical components including a battery (not shown), a power supply (not shown), and connectors for connecting the base module 340 to the mid-section main module 342. The user interface 134 includes a screen 330, buttons 332, knobs 334, and other controls for interaction with the delivery system 100.

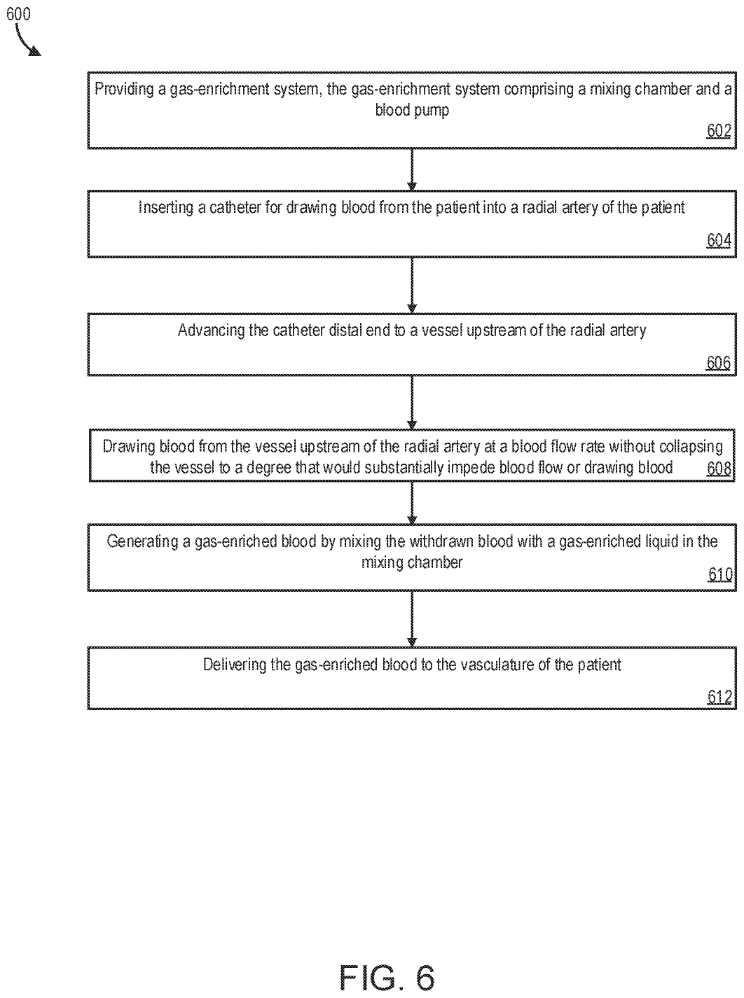

FIG. 6 shows a flow diagram of a process 600 for gas-enrichment, e.g., SSO₂, therapy delivery by one or more of the systems described herein, such as in relation to FIG. 1A-FIG. 5. The process 600 for delivering gas-enriched blood within a vasculature of a patient includes providing (602) a gas-enrichment system, the gas-enrichment system comprising a mixing chamber and a blood pump. The process 600 includes inserting (604) a catheter for drawing blood from the patient into a radial artery of the patient. The process 600 includes advancing (606) the catheter distal end to a vessel upstream of the radial artery. The process 600 includes drawing (608) blood from the vessel upstream of the radial artery at a blood flow rate without collapsing the vessel to a degree that would substantially impede blood flow or drawing blood. The process 600 includes generating (610) a gas-enriched blood by mixing the withdrawn blood with a gas-enriched liquid in the mixing chamber. The process 600 includes delivering (612) the gas-enriched blood to the vasculature of the patient.

In some implementations, the catheter is advanced to a vessel upstream of the radial artery and the blood is drawn from the vessel upstream of the radial artery. For example, the catheter is advanced to the subclavian artery. In some implementations, the blood flow rate is a blood flow rate or predetermined blood flow rate of 10-500 ml/min, 30-300, or 50-150. In some implementations, an inner diameter and length of the catheter are sufficient to support a blood flow rate or predetermined blood flow rate of 50-150 ml/min while avoiding a pressure drop that would cause pump cavitation. In some implementations, the outer diameter is 6-7 French, the length is 10-100 cm, and the pressure drop is from 0 mmHG to negative 100) mmHG.

In some implementations, inserting the catheter into a radial artery of the patient includes, for process 600, accessing a brachial, axillary or subclavian artery of the patient through the radial artery. In some implementations, the process 600 includes advancing the catheter into the brachial, axillary or subclavian artery for drawing the blood from one or more of said arteries.

In some implementations, inserting, for the process 600, the catheter into the vasculature of the patient includes inserting a sheath into the vasculature of the patient, the sheath configured to support the catheter in the vasculature of the patient. In some implementations, the process 600 includes inserting the catheter into the sheath.

In some implementations, the sheath comprises a braided wire and a plastic liner over the braided wire. In some implementations, the sheath is between 50-100 centimeters in length. In some implementations, inserting the catheter into a radial artery of the patient, for process 600, includes advancing the catheter into the radial artery or a vessel upstream of the radial artery of the patient until distal band on the catheter aligns with a predetermined location in the vasculature of the patient.

In some implementations, the process 600 includes controlling a draw rate of the catheter. Controlling the draw rate in process 600 includes determining a maximum draw rate based on a size of the radial artery or vessel upstream from the radial artery. Controlling the draw rate in process 600 includes determining a minimum draw rate and a draw pressure based on a pump flow requirement of a pump configured to draw the blood from the radial artery or a vessel upstream of the radial artery. Controlling the draw rate in process 600 includes controlling the draw rate to be between the maximum draw rate and the minimum draw rate. In some implementations, the maximum draw rate is a draw rate above which would cause a collapse of the radial artery or a vessel upstream of the radial artery to a degree that would substantially impede blood flow or drawing blood. In some implementations, the draw rate is a function of a length of the catheter for process 600. In some implementations, the catheter can be configured for a minimum draw rate of 100 milliliters (mL) per minute and wherein the draw pressure is at least 50 millimeters per Mercury (mmHg).

The process 600 may include measuring the draw rate using a flow sensor. The process 600 may include generating an alert in response to measuring, by the flow sensor, that the draw rate is greater than the maximum draw rate or is less than the minimum draw rate.

The process 600 may include measuring the draw pressure using a pressure sensor. The process 600 may include generating an alert in response to measuring, by the pressure sensor, that the draw pressure is greater than a maximum draw pressure. In some implementations, one or more lumens of the catheter comprise a braided pattern. In some implementations, the braided pattern comprises a rectangular cross section. In some implementations, one or more lumens of the catheter each comprise a wall thickness between 0.005 inches-0.015 inches, the wall thickness preventing kinking of the one or more lumens of the catheter. In some implementations, one or more lumens of the catheter comprise an atraumatic tip. In some implementations, the gas-enriched blood is formed in the mixing chamber by mixing the blood withdrawn from the patient with the gas-enriched liquid generated by a gas enrichment chamber. In some implementations, the gas-enriched liquid comprises a supersaturated oxygen liquid. In some implementations, the supersaturated oxygen liquid has an O₂ concentration of 0.1-6 ml O₂/ml liquid (STP). In some implementations, the gas-enriched blood comprises a supersaturated oxygen enriched blood. In some implementations, the supersaturated oxygen enriched blood comprises a supersaturated oxygen enriched blood having a pO₂ of 600-1500 mmHg.

The process 600 may include inserting a second catheter into a second radial artery of the patient for delivering the gas-enriched blood to the vasculature of the patient.

In some implementations, a control signal corresponding to a measured value of one or more physiological parameters may be received and used to control a pump configured to draw blood from the patient and pump the gas-enriched blood for delivery into the patient. A process may include causing the pump to pump blood to and from the gas-enrichment system and the patient based on sending the control signal to the pump. The sensor can include a flow sensor. The one or more physiological parameters may include a flow rate of blood in the vasculature of the patient. In some implementations, the sensors may include a pressure sensor. The one or more physiological parameters may include a pressure of blood in the vasculature of the patient. In some implementations, the process includes sending, by the controller, the control signal to a pump configured to draw blood from the patient and pump the gas-enriched blood for delivery into the patient. The process includes causing, based on sending the control signal or an alert, the pump to increase a pump speed or reduce a pump speed to increase or reduce the amount or rate of blood drawn from the patient and the amount or rate of gas-enriched blood delivered to the patient. In some implementations, generating the control signal or an alert is performed in real-time or near-real time during delivery of the gas-enriched blood to the patient. The delivery of the gas-enriched blood to the patient is not paused during measurement of the one or more physiological parameters. The measurement of the one or more physiological parameters represents a contemporaneous status of the patient for the delivery of the gas-enriched blood to the patient.

In some implementations, receiving one or more signals corresponding to a measured value of the one or more physiological parameters from the sensor includes receiving a series of measured values of the one or more physiological parameters from the sensor. The series of measured values can correspond to a period of time during delivery of the gas-enriched blood to the patient. The process includes determining, based on the series of measured values corresponding to the period of time, whether the value of the one or more physiological parameters is increasing or decreasing over time. The process includes generating, based on determining that the value of the one or more physiological parameters is increasing or decreasing over time, the control signal or alert for increasing or reducing the pump speed or rate or amount of blood drawn or gas-enriched blood delivered to the patient. The entire disclosures of U.S. Pat. No. 6,743,196, U.S. Pat. No. 6,582,387, U.S. Pat. No. 7,820,102 and U.S. Pat. No. 8,246,564 are expressly incorporated herein by reference.

Some implementations of subject matter and operations described in this specification (e.g., process 600) can be implemented in digital electronic circuitry, or in computer software, firmware, or hardware, including the structures disclosed in this specification and their structural equivalents, or in combinations of one or more of them. For example, in some implementations, the processor of the delivery system (e.g., delivery system 100) can be implemented using digital electronic circuitry, or in computer software, firmware, or hardware, or in combinations of one or more of them.

Some implementations described in this specification (e.g., the processor of the delivery system, etc.) can be implemented as one or more groups or modules of digital electronic circuitry, computer software, firmware, or hardware, or in combinations of one or more of them. Although different modules can be used, each module need not be distinct, and multiple modules can be implemented on the same digital electronic circuitry, computer software, firmware, or hardware, or combination thereof.

Some implementations described in this specification can be implemented as one or more computer programs, i.e., one or more modules of computer program instructions, encoded on computer storage medium for execution by, or to control the operation of, data processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing, and grid computing infrastructures.

A computer program (also known as a program, software, software application, script, or code) can be written in any form of programming language, including compiled or interpreted languages, declarative or procedural languages. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed for execution on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and processors of any kind of digital computer. Generally, a processor will receive instructions and data from a read only memory or a random-access memory or both. A computer includes a processor for performing actions in accordance with instructions and one or more memory devices for storing instructions and data. A computer may also include, or be operatively coupled to receive data from or transfer data to, or both, one or more mass storage devices for storing data, e.g., magnetic, magneto optical disks, or optical disks. However, a computer need not have such devices. Devices suitable for storing computer program instructions and data include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks, and CD-ROM and DVD-ROM disks. The processor and the memory can be supplemented by, or incorporated in, special purpose logic circuitry.

To provide for interaction with a user, operations can be implemented on a computer having a display device (e.g., a monitor, or another type of display device) for displaying information to the user and a keyboard and a pointing device (e.g., a mouse, a trackball, a tablet, a touch sensitive screen, or another type of pointing device) by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A computer system may include a single computing device, or multiple computers that operate in proximity or generally remote from each other and typically interact through a communication network. Examples of communication networks include a local area network (“LAN”) and a wide area network (“WAN”), an inter-network (e.g., the Internet), a network comprising a satellite link, and peer-to-peer networks (e.g., ad hoc peer-to-peer networks). A relationship of client and server may arise by virtue of computer programs running on the respective computers and having a client-server relationship to each other.

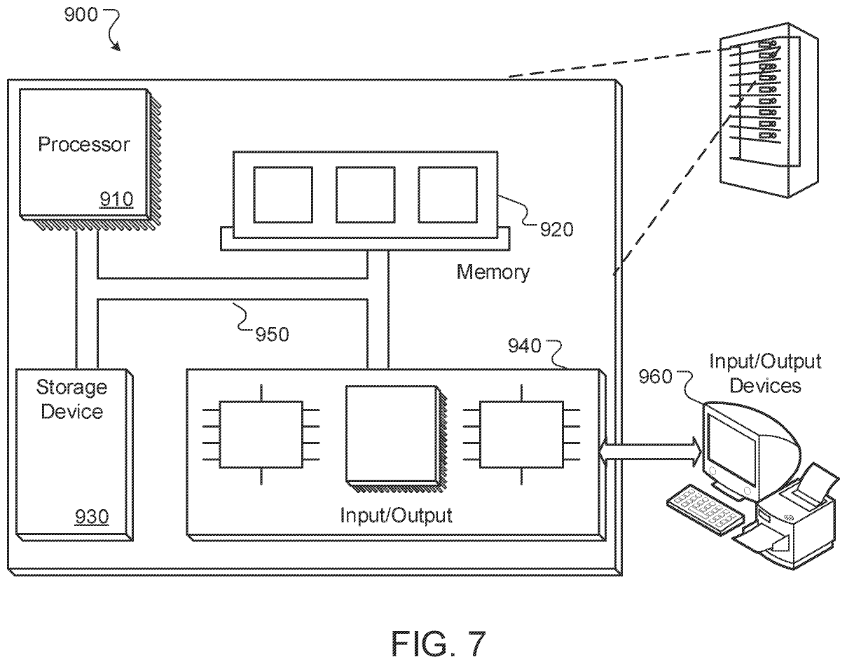

FIG. 7 shows an example computer system 900 that includes a processor 910, a memory 920, a storage device 930 and an input/output device 940. Each of the components 910, 920, 930 and 940 can be interconnected, for example, by a system bus 950. The processor 910 is capable of processing instructions for execution within the system 900. In some implementations, the processor 910 is a single-threaded processor, a multi-threaded processor, or another type of processor. The processor 910 is capable of processing instructions stored in the memory 920 or on the storage device 930. The memory 920 and the storage device 930 can store information within the system 900.

The input/output device 940 provides input/output operations for the system 900. In some implementations, the input/output device 940 can include one or more of a network interface device, e.g., an Ethernet card, a serial communication device, e.g., an RS-232 port, and/or a wireless interface device, e.g., an 802.11 card, a 3G wireless modem, a 4G wireless modem, a 5G wireless modem, etc. In some implementations, the input/output device can include driver devices configured to receive input data and send output data to other input/output devices, e.g., keyboard, printer and display devices 960. In some implementations, mobile computing devices, mobile communication devices, and other devices can be used.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable sub-combination.

A number of embodiments have been described. For example, the detailed description and the accompanying drawings to which it refers are intended to describe some, but not necessarily all, examples or embodiments of the system. The described embodiments are to be considered in all respects only as illustrative and not restrictive. Nevertheless, various modifications may be made without departing from the scope of the data processing system described herein. Accordingly, other embodiments are within the scope of the following claims.

Substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

Information and signals may be represented using any of a variety of different technologies and techniques. For example, data, instructions, commands, information, signals, and symbols that may be referenced throughout the above description may be represented by voltages, currents, electromagnetic waves, magnetic fields or particles, optical fields or particles, or any combination thereof.

The methods, systems, and devices discussed above are examples. Various alternative configurations may omit, substitute, or add various procedures or components as appropriate. Configurations may be described as a process which is depicted as a flow diagram or block diagram. Although each may describe the operations as a sequential process, many of the operations can be performed in parallel or concurrently. In addition, the order of the operations may be rearranged. A process may have additional stages not included in the figure. Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional stages or functions not included in the figure. Furthermore, examples of the methods may be implemented by hardware, software, firmware, middleware, microcode, hardware description languages, or any combination thereof. When implemented in software, firmware, middleware, or microcode, the program code or code segments to perform the tasks may be stored in a non-transitory processor-readable medium such as a storage medium. Processors may perform the described tasks.

Components, functional or otherwise, shown in the figures and/or discussed herein as being connected or communicating with each other are communicatively coupled. That is, they may be directly or indirectly connected to enable communication between them.

As used herein, including in the claims, “and” as used in a list of items prefaced by “at least one of” indicates a disjunctive list such that, for example, a list of “at least one of A, B, and C” means A or B or C or AB or AC or BC or ABC. (i.e., A and B and C), or combinations with more than one feature (e.g., AA, AAB, ABBC, etc.). As used herein, including in the claims, unless otherwise stated, a statement that a function or operation is “based on” an item or condition means that the function or operation is based on the stated item or condition and may be based on one or more items and/or conditions in addition to the stated item or condition.

Having described several example configurations, various modifications, alternative constructions, and equivalents may be used without departing from the disclosure. For example, the above elements may be components of a larger system, wherein other rules may take precedence over or otherwise modify the application of the invention. Also, a number of operations may be undertaken before, during, or after the above elements are considered. Also, technology evolves and, thus, many of the elements are examples and do not bound the scope of the disclosure or claims. Accordingly, the above description does not bound the scope of the claims. Further, more than one invention may be disclosed.

Other embodiments are within the scope of the invention. For example, due to the nature of software, functions described above can be implemented using software, hardware, firmware, hardwiring, or combinations of any of these. Features implementing functions may also be physically located at various locations, including being distributed such that portions of functions are implemented at different physical locations.

The claims should not be read as limited to the described order or elements unless stated to that effect. It should be understood that various changes in form and detail may be made by one of ordinary skill in the art without departing from the spirit and scope of the appended claims. All implementations that come within the spirit and scope of the following claims and equivalents thereto are claimed.

1. A method for delivering gas-enriched blood within a vasculature of a patient, the method comprising:
providing a gas-enrichment system the gas-enrichment system comprising a mixing chamber and a blood pump;
inserting a catheter for drawing blood from the patient into a radial artery of the patient;
drawing blood from the radial artery or from a vessel upstream of the radial artery at a blood flow rate without collapsing the artery or vessel to a degree that would substantially impede drawing blood;
generating a gas-enriched blood by mixing withdrawn blood with a gas-enriched liquid in the mixing chamber; and
delivering the gas-enriched blood to the vasculature of the patient.

2. The method of claim 1, wherein the catheter is advanced to the vessel upstream of the radial artery and the blood is drawn from the vessel upstream of the radial artery.

3. (canceled)

4. The method of claim 1, wherein an inner diameter and a length of the catheter are sufficient to support a predetermined blood flow rate of 50-150 ml/min while avoiding a pressure drop that would cause pump cavitation.

5. The method of claim 4, wherein the inner diameter is 6-7 French, the length is 10 to 100 cm, and the pressure drop is from 0 mmHG to at least negative 50 mmHG.

6. The method of claim 1, wherein inserting the catheter into a radial artery of the patient comprises:
accessing a subclavian artery of the patient through the radial artery; and
advancing the catheter into the subclavian artery for drawing the blood from the subclavian artery.

7.-9. (canceled)

10. The method of claim 1, wherein inserting the catheter into a radial artery of the patient comprises:
advancing the catheter into the radial artery of the patient until distal band on the catheter alignment with a predetermined location in the vasculature of the patient.

11. The method of claim 1, further comprising:
controlling a draw rate of the catheter, wherein the controlling comprises:
determining a maximum draw rate based on a size of the radial artery;
determining a minimum draw rate and a draw pressure based on a pump flow requirement of a pump configured to draw the blood from the radial artery; and
controlling the draw rate to be between the maximum draw rate and the minimum draw rate.

12.-13. (canceled)

14. The method of claim 11, wherein the minimum draw rate is 100 milliliters (ml) per minute and wherein the draw pressure is at least 50 millimeters per Mercury (mmHg).

15. The method of claim 11, further comprising:
measuring the draw rate using a flow sensor; and
generating an alert in response to measuring, by the flow sensor, that the draw rate is greater than the maximum draw rate or is less than the minimum draw rate.

16. The method of claim 11, further comprising:
measuring the draw pressure using a pressure sensor; and
generating an alert in response to measuring, by the pressure sensor, that the draw pressure is greater than a maximum draw pressure.

17.-20. (canceled)

21. The method of claim 1, wherein the gas-enriched blood is formed in the mixing chamber by mixing the blood withdrawn from the patient with the gas-enriched liquid generated by a gas enrichment chamber.

22.-23. (canceled)

24. The method of claim 1, wherein the gas-enriched blood comprises a supersaturated oxygen enriched blood.

25. The method of claim 24, wherein the supersaturated oxygen enriched blood comprises a supersaturated oxygen enriched blood having a pO2 of 600-1500 mmHg.

26. The method of claim 1, further comprising:
inserting a second catheter into a second radial artery of the patient for delivering the gas-enriched blood to the vasculature of the patient.

27. The method of claim 1, further comprising:
measuring a blood pressure in the radial artery or a vessel upstream of the radial artery using one or more pressure sensors, wherein a controller of the gas-enrichment system receives a signal from the one or more pressure sensors.

28. The method of claim 27 wherein the controller generates an alert in response to receiving a signal from the pressure sensor indicating a blood pressure or change in blood pressure that exceeds a threshold or is below a threshold.

29. The method of claim 27, wherein the controller controls a pump to adjust a blood draw flow rate in response to receiving a signal from the pressure sensor indicating a blood pressure or change in blood pressure that exceeds a threshold or is below a threshold.

30. (canceled)

31. The method of claim 1, wherein drawing blood without collapsing the artery or vessel to a degree that would substantially impede drawing blood comprises preventing a collapse that would result in more than 5-10% reduction in cross-sectional area of an artery or vessel.

32.-33. (canceled)

34. A system for delivering gas-enriched blood within a vasculature of a patient, the system comprising:
a blood circuit, comprising:
a pump configured to circulate blood in the blood circuit;
a mixing chamber configured to mix blood of the patient with a gas-enriched liquid to form a gas-enriched blood;
a catheter; and
a draw line coupled to the mixing chamber and configured to connect the catheter to the mixing chamber;
wherein the catheter is configured to be inserted into a radial artery of the patient, the catheter comprising one or more lumens configured to draw the blood from the radial artery or from a vessel upstream of the radial artery at a blood flow rate without collapsing the artery or vessel to a degree that would substantially impede drawing blood and send the blood to the mixing chamber.

35.-104. (canceled)

105. The method of claim 1, wherein blood is drawn at a blood flow rate of 10-500 mL/min and drawing blood without collapsing the artery or vessel to a degree that would substantially impede drawing blood comprises preventing a reduction of blood flow over a threshold percentage of about 10-15%.

106.-107. (canceled)
